(19) United States
(12) Patent Application Publication
(10) Pub. No.: US 2022/0031148 A1
(43) Pub. Date: Feb. 3, 2022

(54) ENDOSCOPIC SYSTEMS, DEVICES, AND METHODS

(71) Applicant: Bio-Medical Engineering (HK) Limited, Hong Kong (CN)

(72) Inventors: Chung Kwong Yeung, Hong Kong (CN); Kan Sang Mok, Hong Kong (CN); Wing Fai Lam, Hong Kong (CN); Ka Man Chan, Hong Kong (CN); King Ng, Hong Kong (CN); Yau Sik Tang, Hong Kong (CN)

(21) Appl. No.: 17/503,172

(22) Filed: Oct. 15, 2021

Related U.S. Application Data
(63) Continuation of application No. PCT/CN2019/082814, filed on Apr. 16, 2019.

(51) Int. Cl. A61B 1/00; A61B 1/31

(52) U.S. Cl. CPC A61B 1/00082; A61B 1/31

(57) ABSTRACT

Example embodiments relate to endoscopic systems, devices, and methods. The system may include a main assembly having a main assembly body and expandable member. Main assembly body may include a proximal region, distal region, and central region having a pressure opening for providing positive pressure. Expandable member may have a tubular structure with interior channel, inward facing interior surface, outward facing exterior surface, and first and second ends. Interior channel may house the central region of the main assembly body. First end of the expandable member may be secured to the proximal region of the main assembly body. Second end of the expandable member may be secured to the distal region of the main assembly body. When the pressure opening provides positive pressure, the tubular structure of the expandable member between the first and second ends of the expandable member may expand outwardly away from the central region.

### CROSS-REFERENCE TO RELATED APPLICATIONS

This application is a continuation application of International Application No. PCT/CN2019/082814 filed Apr. 16, 2019, the contents of which is hereby expressly incorporated by reference in its entirety, including the contents and teachings of any references contained therein.

### BACKGROUND

The present disclosure relates generally to endoscopic systems, devices, and methods, and more specifically, relates to systems and devices for use in performing endoluminal procedures, including diagnostic and therapeutic procedures, and methods of configuring and using such systems and devices.

Presently, colorectal cancer is the third most commonly diagnosed cancer and also the third leading cause of cancer-related deaths worldwide. If diagnosed at a sufficiently early stage, however, the survival rate of patients suffering from colorectal cancer may reach upwards of ninety percent.

Conventional optical colonoscopy is the most widely accepted and used procedure for colorectal screening. In general, conventional optical colonoscopy involves the insertion of a colonoscope through the colon of a patient.

### BRIEF SUMMARY

Despite recent developments in modern medical science and technology, it is recognized in the present disclosure that one or more problems are encountered in colonoscopy-related diagnostic and therapeutic technologies and methodologies, including those described in the present disclosure.

Present example embodiments relate generally to systems, devices, and methods for addressing one or more problems in diagnostic and therapeutic systems, devices, and methods, including those described above and herein.

In an exemplary embodiment, an endoscopic system is described. The endoscopic system may include a main assembly. The main assembly may include a main assembly body and an expandable member. The main assembly body may be cylindrical in shape and have a proximal region, a distal region, and a central region between the proximal and distal regions. The central region may have one or more first pressure openings formed through an exterior surface of the central region. The one or more first pressure openings may be configurable to provide an outward positive pressure. The proximal region, central region, and distal region are collectively formed in such a way as to have a common central axis. The expandable member may be secured to the main assembly body. The expandable member may include a tubular structure with an interior channel. The expandable member may include an inward facing interior surface forming the interior channel. The expandable member may include an outward facing exterior surface opposite to the inward facing interior surface. The expandable member may include a first end corresponding to an end of the tubular structure of the expandable member. The expandable member may include a second end corresponding to another end of the tubular structure of the expandable member. The interior channel of the expandable member may house the central region of the main assembly body in such a way that the one or more first pressure openings face the inward facing interior surface of the expandable member. The first end of the expandable member may be secured to the proximal region of the main assembly body. The second end of the expandable member may be secured to the distal region of the main assembly body. When the one or more first pressure openings provide an outward positive pressure, the tubular structure of the expandable member between the first and second ends of the expandable member may be configured to expand outwardly away from the central region of the main assembly body.

In example embodiments described above and in the present disclosure, the first end of the expandable member may be secured to the proximal region of the main assembly body in a hermetically sealed manner. The second end of the expandable member may be secured to the distal region of the main assembly body in a hermetically sealed manner. The first end of the expandable member may or may not be secured to the proximal region of the main assembly body via an overmolding process. The second end of the expandable member may or may not be secured to the distal region of the main assembly body via an overmolding process. The proximal region, central region, and distal region may or may not be formed as a unitary body. The first end of the expandable member may or may not be secured to a first ring-shaped member, and the first end of the expandable member may or may not be secured to the proximal region of the main assembly body by securing the first ring-shaped member to the proximal region of the main assembly body. The second end of the expandable member may or may not be secured to a second ring-shaped member, and the second end of the expandable member may or may not be secured to the distal region of the main assembly body by securing the second ring-shaped member to the distal region of the main assembly body. The common central axis of the proximal region, central region, and distal region may correspond to the central axis of the cylindrically shaped main assembly body. Each of the central region, proximal region, and distal region may or may not be cylindrical in shape. The tubular structure of the expandable member may or may not be a single thin-walled tubular structure. The endoscopic system may further include a pressure source connected to the one or more first pressure openings. In such embodiment, when the first and second ends of the expandable member are secured to the proximal and distal regions of the main assembly body, respectively, the pressure source and the one or more first pressure openings may be collectively configurable to cooperate with the expandable member and the exterior surface of the central region of the main assembly body to form a hermetically sealed chamber. When the expandable member is not expanded outwardly by the outward positive pressure of the one or more first pressure openings, at least a portion of the inward facing interior surface of the expandable member may or may not be in contact with at least a portion of the exterior surface of the central region of the main assembly body. When the one or more first pressure openings provide an outward positive pressure exceeding a threshold value, the tubular structure of the expandable member between the first and second ends of the expandable member may be configured to expand outwardly away from the exterior surface of the central region of the main assembly body in such a way as to form a hollow toroidal-shaped body surrounding the central region of the main assembly body. Such hollow toroidal-shaped body may include a substantially D-shaped cross-section with a flat inner side and a curved side. Such flat inner side may correspond to the exterior surface of the central region of the main assembly body and such curved side may correspond to at least a portion of the expandable member. The expandable member may be formed from a rectangular-shaped sheet of expandable membrane having a first side, second side, third side opposite to the first side, and fourth side opposite to the second side. In such embodiment, the first and second ends of the expandable member corresponding to the first and third sides, wherein at least a portion of the second and fourth sides are secured to the exterior surface of the central region of the main assembly body. The endoscopic system may further include an elongated tubular member having proximal and distal ends. Such distal end of the elongated tubular member may be secured to the proximal region of the main assembly body.

In another exemplary embodiment, an endoscopic system is described. The endoscopic system may include a main assembly. The main assembly may include a main assembly body and a first expandable member. The main assembly body may include a proximal region, an intermediate region, a first central region between the proximal and intermediate regions, a distal region, and a second central region between the intermediate and distal regions. The first central region may include one or more first pressure openings formed through an exterior surface of the first central region. The second central region may include one or more second pressure openings formed through an exterior surface of the second central region. The proximal region, first central region, intermediate region, second central region, and distal region may be collectively formed in such a way as to have a common central axis. The first expandable member may be secured to the main assembly body. The first expandable member may include a tubular structure with a first interior channel. The first expandable member may include an inward facing first interior surface forming the first interior channel. The first expandable member may include an outward facing first exterior surface opposite to the inward facing first interior surface. The first expandable member may include a first end corresponding to an end of the tubular structure of the first expandable member. The first expandable member may include a second end corresponding to another end of the tubular structure of the first expandable member. The first interior channel of the first expandable member may house the first central region of the main assembly body in such a way that the one or more first pressure openings face the inward facing first interior surface of the first expandable member. The first end of the first expandable member may be secured to the proximal region of the main assembly body. The second end of the first expandable member may be secured to the intermediate region of the main assembly body. When the one or more first pressure openings provide an outward positive pressure, the tubular structure of the first expandable member between the first and second ends of the first expandable member may be configured to expand outwardly away from the first central region of the main assembly body.

In example embodiments described above and in the present disclosure, the main assembly may further include a second expandable member secured to the main assembly body. The second expandable member may include a tubular structure with a second interior channel. The second expandable member may include an inward facing second interior surface forming the second interior channel. The second expandable member may include an outward facing second exterior surface opposite to the inward facing second interior surface. The second expandable member may include a first end corresponding to an end of the tubular structure of the second expandable member. The second expandable member may include a second end corresponding to another end of the tubular structure of the second expandable member. The second interior channel of the second expandable member may house the second central region of the main assembly body in such a way that the one or more second pressure openings face the inward facing second interior surface of the second expandable member. The first end of the second expandable member may be secured to the intermediate region of the main assembly body. The second end of the second expandable member may be secured to the distal region of the main assembly body. When the one or more second pressure openings provide an outward positive pressure, the tubular structure of the second expandable member between the first and second ends of the second expandable member may be configured to expand outwardly away from the second central region of the main assembly body. When the one or more first pressure openings provide an outward positive pressure exceeding a threshold value, the tubular structure of the first expandable member between the first and second ends of the first expandable member may be configured to expand outwardly away from the exterior surface of the first central region of the main assembly body in such a way as to form a hollow toroidal-shaped body surrounding the first central region of the main assembly body. In such embodiment, the hollow toroidal-shaped body may include a substantially D-shaped cross-section with a flat inner side and a curved side. Such flat inner side may correspond to the exterior surface of the first central region of the main assembly body and such curved side may correspond to at least a portion of the first expandable member. When the one or more second pressure openings provide an outward positive pressure exceeding a threshold value, the tubular structure of the second expandable member between the first and second ends of the second expandable member may be configured to expand outwardly away from the exterior surface of the second central region of the main assembly body in such a way as to form a hollow toroidal-shaped body surrounding the second central region of the main assembly body. In such embodiment, the hollow toroidal-shaped body may include a substantially D-shaped cross-section with a flat inner side and a curved side. Such flat inner side may correspond to the exterior surface of the second central region of the main assembly body and such curved side may correspond to at least a portion of the second expandable member. The endoscopic system may further include an elongated tubular member having proximal and distal ends. Such distal end of the elongated tubular member may be secured to the proximal region of the main assembly body. The first expandable member may be formed from a rectangular-shaped sheet of expandable membrane having a first side, second side, third side opposite to the first side, and fourth side opposite to the second side. Such first and second ends of the first expandable member may correspond to the first and third sides. At least a portion of such second and fourth sides may be secured to the exterior surface of the first central region of the main assembly body.

In another exemplary embodiment, an endoscopic system is described. The endoscopic system may include a first main assembly and second main assembly. The first main assembly may include a first main assembly body and a first expandable member. The first main assembly body may include a first proximal region, a first intermediate region, a first proximal central region between the first proximal region and first intermediate region, a first distal region, and a first distal central region between the first intermediate region and first distal region. The first proximal central region may include one or more first pressure openings formed through an exterior surface of the first proximal central region. The first proximal region, first proximal central region, first intermediate region, first distal central region, and first distal region may be collectively formed in such a way as to have a common first central axis. The first expandable member may be secured to the first main assembly body. The first expandable member may include a tubular structure with a first interior channel. The first expandable member may include an inward facing first interior surface forming the first interior channel. The first expandable member may include an outward facing first exterior surface opposite to the inward facing first interior surface. The first expandable member may include a first end corresponding to an end of the tubular structure of the first expandable member. The first expandable member may include a second end corresponding to another end of the tubular structure of the first expandable member. The first interior channel of the first expandable member may house the first proximal central region of the first main assembly body in such a way that the one or more first pressure openings face the inward facing first interior surface of the first expandable member. The first end of the first expandable member may be secured to the first proximal region of the first main assembly body. The second end of the first expandable member may be secured to the first intermediate region of the first main assembly body. When the one or more first pressure openings provide an outward positive pressure, the tubular structure of the first expandable member between the first and second ends of the first expandable member may be configured to expand outwardly away from the first proximal central region of the first main assembly body. The second main assembly may be adjustably positionable relative to the first main assembly. The first and second main assemblies may be configured in such a way that a distance between the first and second main assemblies is adjustable. The second main assembly may include a second main assembly body and a second expandable member. The second main assembly body may include a second proximal region, a second intermediate region, a second proximal central region between the second proximal region and second intermediate region, a second distal region, and a second distal central region between the second intermediate region and second distal region. The second proximal central region may include one or more second pressure openings formed through an exterior surface of the second proximal central region. The second proximal region, second proximal central region, second intermediate region, second distal central region, and second distal region may be collectively formed in such a way as to have a common second central axis. The second expandable member may be secured to the second main assembly body. The second expandable member may include a tubular structure with a second interior channel. The second expandable member may include an inward facing second interior surface forming the second interior channel. The second expandable member may include an outward facing second exterior surface opposite to the inward facing second interior surface. The second expandable member may include a first end corresponding to an end of the tubular structure of the second expandable member. The second expandable member may include a second end corresponding to another end of the tubular structure of the second expandable member. The second interior channel of the second expandable member may house the second proximal central region of the second main assembly body in such a way that the one or more second pressure openings face the inward facing second interior surface of the second expandable member. The first end of the second expandable member may be secured to the second proximal region of the second main assembly body. The second end of the second expandable member may be secured to the second intermediate region of the second main assembly body. When the one or more second pressure openings provide an outward positive pressure, the tubular structure of the second expandable member between the first and second ends of the second expandable member may be configured to expand outwardly away from the second proximal central region of the second main assembly body.

In example embodiments described above and in the present disclosure, the first main assembly may include a third expandable member secured to the first main assembly body. The third expandable member may include a tubular structure with a third interior channel. The third expandable member may include an inward facing third interior surface forming the third interior channel. The third expandable member may include an outward facing third exterior surface opposite to the inward facing third interior surface. The third expandable member may include a first end corresponding to an end of the tubular structure of the third expandable member. The third expandable member may include a second end corresponding to another end of the tubular structure of the third expandable member. The first distal central region of the first main assembly body may include one or more third pressure openings formed through an exterior surface of the first distal central region. The third interior channel of the third expandable member may house the first distal central region of the first main assembly body in such a way that the one or more third pressure openings face the inward facing third interior surface of the third expandable member. The first end of the third expandable member may be secured to the first intermediate region of the first main assembly body. The second end of the third expandable member may be secured to the first distal region of the first main assembly body. When such one or more third pressure openings provide an outward positive pressure, the tubular structure of the third expandable member between the first and second ends of the third expandable member may be configured to expand outwardly away from the first distal central region of the first main assembly body. When the one or more first pressure openings provide an outward positive pressure exceeding a threshold value, the tubular structure of the first expandable member between the first and second ends of the first expandable member may be configured to expand outwardly away from the exterior surface of the first proximal central region of the first main assembly body in such a way as to form a hollow toroidal-shaped body surrounding the first proximal central region of the main assembly body. When the one or more second pressure openings provide an outward positive pressure exceeding a threshold value, the tubular structure of the second expandable member between the first and second ends of the second expandable member may be configured to expand outwardly away from the exterior surface of the second proximal central region of the second main assembly body in such a way as to form a hollow toroidal-shaped body surrounding the second proximal central region of the second main assembly body. The first distal region may include an image capturing assembly. The first intermediate region may include one or more fourth pressure openings. Such one or more fourth pressure openings may be configurable to provide an inward negative pressure. The first end of the first expandable membrane sheet may or may not be secured to the first proximal region of the first main assembly body via an overmolding process. The second end of the first expandable membrane sheet may or may not be secured to the first intermediate region of the first main assembly body via an overmolding process. The endoscopic system may further include an extendible section. Such extendible section may include an extendible section body. Such extendible section body may include a first extendible section end secured to the first main assembly and a second extendible section end secured to the second main assembly. Such extendible section may be configurable to adjust an overall length of the extendible section body between the first extendible section end and the second extendible section end. The distance between the first and second main assemblies may be changed by configuring the extendible section to change the overall length of the extendible section body.

### BRIEF DESCRIPTION OF THE DRAWINGS

For a more complete understanding of the present disclosure, example embodiments, and their advantages, reference is now made to the following description taken in conjunction with the accompanying drawings, in which like reference numbers indicate like features, and:

FIG. 1A is an illustration of a perspective view of an example embodiment of an endoscopic system having expandable members that are not expanded (in an unexpanded state);

FIG. 1B is an illustration of a side view of an example embodiment of an endoscopic system without expandable members;

FIG. 1C is an illustration of a side view of an example embodiment of an endoscopic system having expandable members that are not expanded (in an unexpanded state);

FIG. 1D is an illustration of a perspective view of an example embodiment of an endoscopic system having an expandable member that is not expanded (in an unexpanded state);

FIG. 1E is an illustration of a side view of an example embodiment of an endoscopic system having an expandable member that is not expanded (in an unexpanded state);

FIG. 2A is an illustration of a perspective view of an example embodiment of an endoscopic system having expandable members that are expanded (in an expanded state);

FIG. 2B is an illustration of a side view of an example embodiment of an endoscopic system having expandable members that are expanded (in an expanded state);

FIG. 3A is an illustration of a perspective view of an example embodiment of an endoscopic system having expandable members that are expanded (in an expanded state);

FIG. 3B is an illustration of a side view of an example embodiment of an endoscopic system without expandable members;

FIG. 3C is an illustration of a side view of an example embodiment of an endoscopic system having expandable members that are not expanded (in an unexpanded state);

FIG. 3D is an illustration of a side view of an example embodiment of an endoscopic system having expandable members that are not expanded (in an unexpanded state) and extendible section extended (in an extended state);

FIG. 3E is an illustration of a side view of an example embodiment of an endoscopic system having expandable members that are not expanded (in an unexpanded state) and extendible section in a bending configuration;

FIG. 4A is an illustration of a perspective view of an example embodiment of an expandable member in the form of a rectangular sheet, or the like;

FIG. 4B is an illustration of a perspective view of an example embodiment of an expandable member having a tubular (or cylindrical) structure or shape;

FIG. 4C is an illustration of a perspective view of an example embodiment of an expandable member having a tubular (or cylindrical) structure or shape;

FIG. 4D is an illustration of a perspective view of an example embodiment of an expandable member having a tubular (or cylindrical) structure or shape and having ends secured to a ring-shaped member;

FIG. 4E is an illustration of a top (or bottom) view of an example embodiment of an expandable member having a tubular (or cylindrical) structure or shape;

FIG. 5A is an illustration of a cross-sectional view of an example embodiment of an endoscopic system without expandable members;

FIG. 5B is an illustration of a cross-sectional view of an example embodiment of an endoscopic system with expandable members that are not expanded (in an unexpanded state);

FIG. 5C is an illustration of a cross-sectional view of an example embodiment of an endoscopic system with expandable members that are expanded (in an expanded state);

FIG. 6A is an illustration of a cross-sectional view of an example embodiment of an endoscopic system without expandable members;

FIG. 6B is an illustration of a cross-sectional view of an example embodiment of an endoscopic system with expandable members that are not expanded (in an unexpanded state);

FIG. 6C is an illustration of a cross-sectional view of an example embodiment of an endoscopic system with expandable members that are expanded (in an expanded state);

FIG. 7A is an illustration of a cross-sectional view of an example embodiment of an endoscopic system without expandable members;

FIG. 7B is an illustration of a cross-sectional view of an example embodiment of an endoscopic system with expandable members that are not expanded (in an unexpanded state); and

FIG. 7C is an illustration of a cross-sectional view of an example embodiment of an endoscopic system with expandable members that are expanded (in an expanded state).

Although similar reference numbers may be used to refer to similar elements in the figures for convenience, it can be appreciated that each of the various example embodiments may be considered to be distinct variations.

Example embodiments will now be described with reference to the accompanying drawings, which form a part of the present disclosure, and which illustrate example embodiments which may be practiced. As used in the present disclosure and the appended claims, the terms “example embodiment,” “exemplary embodiment,” and “present embodiment” do not necessarily refer to a single embodiment, although they may, and various example embodiments may be readily combined and/or interchanged without departing from the scope or spirit of example embodiments. Furthermore, the terminology as used in the present disclosure and the appended claims is for the purpose of describing example embodiments only and is not intended to be limitations. In this respect, as used in the present disclosure and the appended claims, the term “in” may include “in” and “on,” and the terms “a,” “an” and “the” may include singular and plural references. Furthermore, as used in the present disclosure and the appended claims, the term “by” may also mean “from,” depending on the context. Furthermore, as used in the present disclosure and the appended claims, the term “if” may also mean “when” or “upon,” depending on the context. Furthermore, as used in the present disclosure and the appended claims, the words “and/or” may refer to and encompass any and all possible combinations of one or more of the associated listed items.

### DETAILED DESCRIPTION

It is recognized in the present disclosure that one or more problems are encountered in endoscopic technologies and methodologies, including those described above and in the present disclosure. For example, conventional optical colonoscopy generally involves an insertion of a colonoscope through a colon of a patient, and requires forceful manual pushing of the colonoscope against the interior luminal walls forming the colon cavity at flexural or looping/bending sections of the colon during insertion, which generally results in severe discomfort and/or pain to the patient. Furthermore, the retracting and/or removal of the colonoscope, including the traversing of the colonoscope through the flexural and/or looping/bending sections of the colon of the patient, may also give rise to discomfort and/or pain to the patient.

Recent developments in diagnostic procedures and devices have attempted to address the aforementioned problem through the use of a miniaturized wireless capsule having an integrated camera. To perform the diagnostic procedure, the miniaturized capsule is orally introduced into a patient, and the miniaturized capsule passively navigates via peristalsis along the gastrointestinal tract in a painless manner. It is recognized in the present disclosure, however, that while such recent developments address the issue of discomfort and pain to patients, such recent developments are not without its own problems and limitations. For example, the in vivo monitoring of the gastrointestinal tract by such miniaturized capsules is in fact performed in a non-controlled and very slow manner since locomotion of the miniaturized capsule through the gastrointestinal tract occurs via peristalsis. Furthermore, while a miniaturized capsule generally takes between about 20 to 36 hours to travel through an entire gastrointestinal tract, current power capacity and consumption of such miniaturized capsules are only capable of roughly about eight hours of operation. Accordingly, not all of the gastrointestinal tract can be imaged and/or monitored using such technology. Furthermore, such miniaturized capsules are merely capable of performing imaging/diagnosing procedures, and not capable of performing therapeutic/surgical procedures, such as a removing of polyps, obtaining biopsy samples, and/or the like.

Systems, devices, and methods, including those for use in endoscopy and colonoscopy, are described in the present disclosure for addressing one or more problems of known systems, devices, and methods, including those described above and in the present disclosure. It is to be understood that the principles described in the present disclosure may be applied outside of the context of endoscopy and colonoscopy, such as performing diagnostic procedures, surgical or therapeutic procedures, scientific experiments, and/or other procedures in the same and/or other environments, cavities, and/or organs not described in the present disclosure without departing from the teachings of the present disclosure.

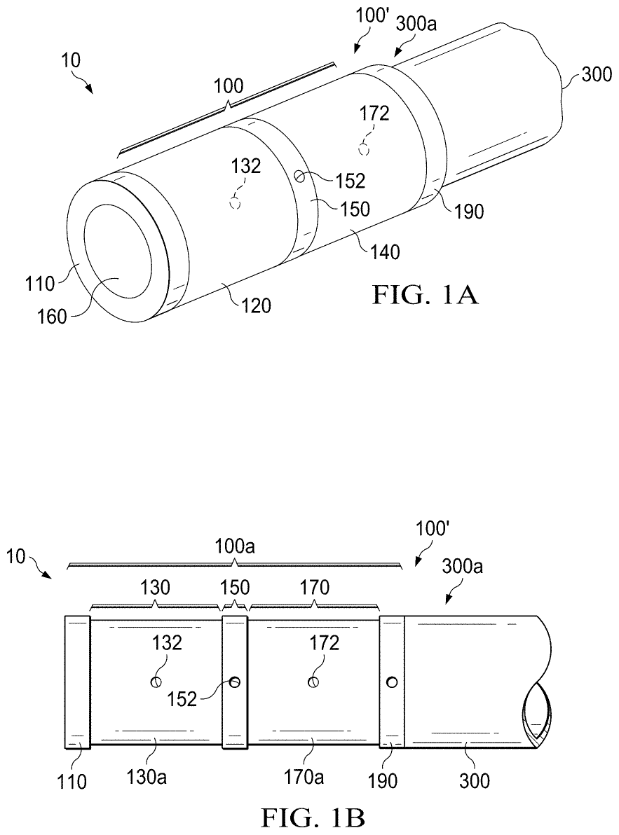

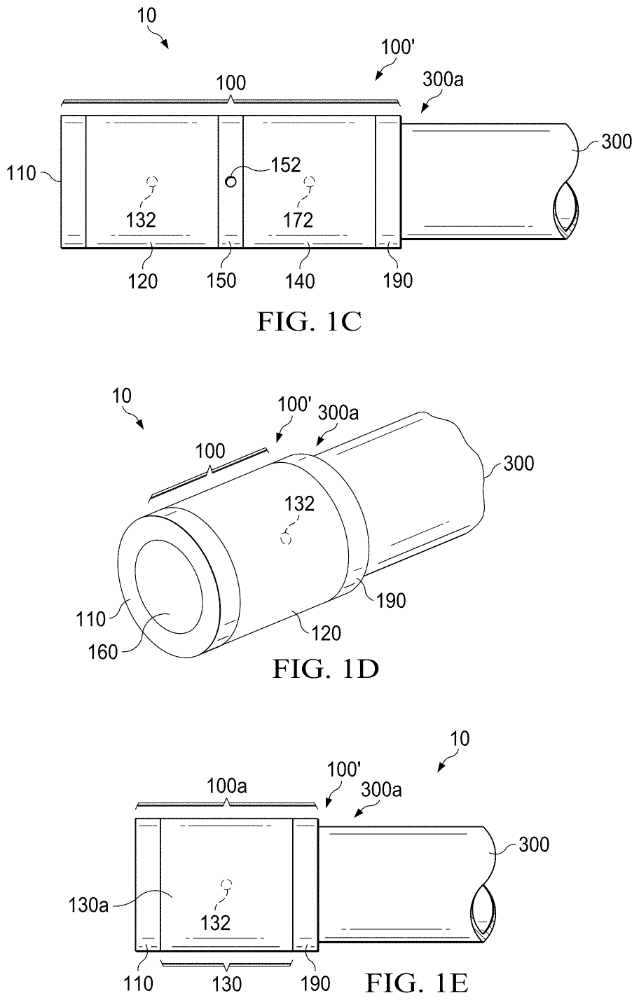

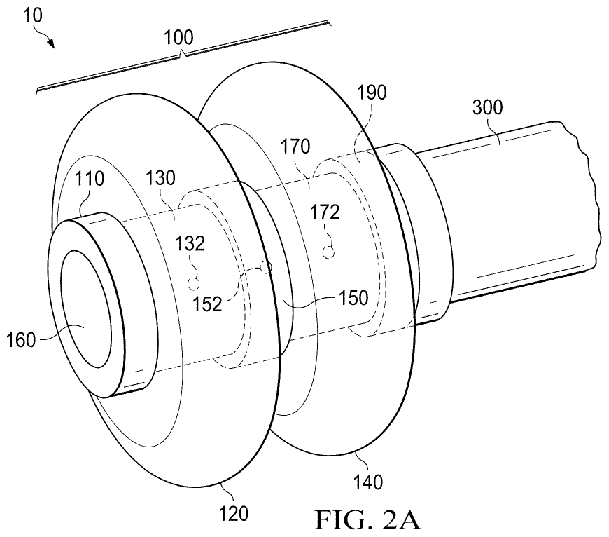

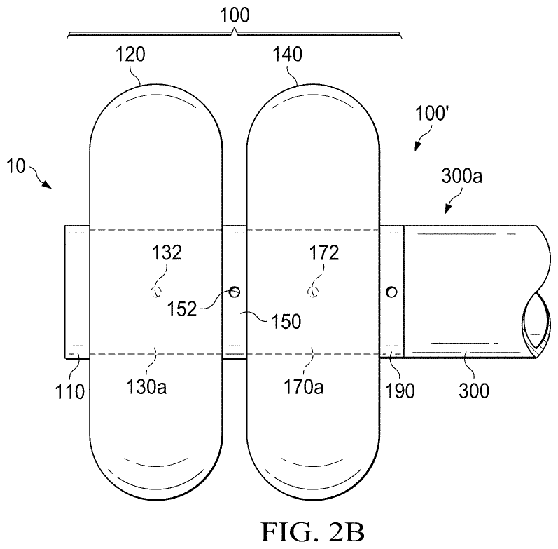

The Endoscopic System (e.g., the Endoscopic System 10 Illustrated in at Least FIGS. 1 and 2).

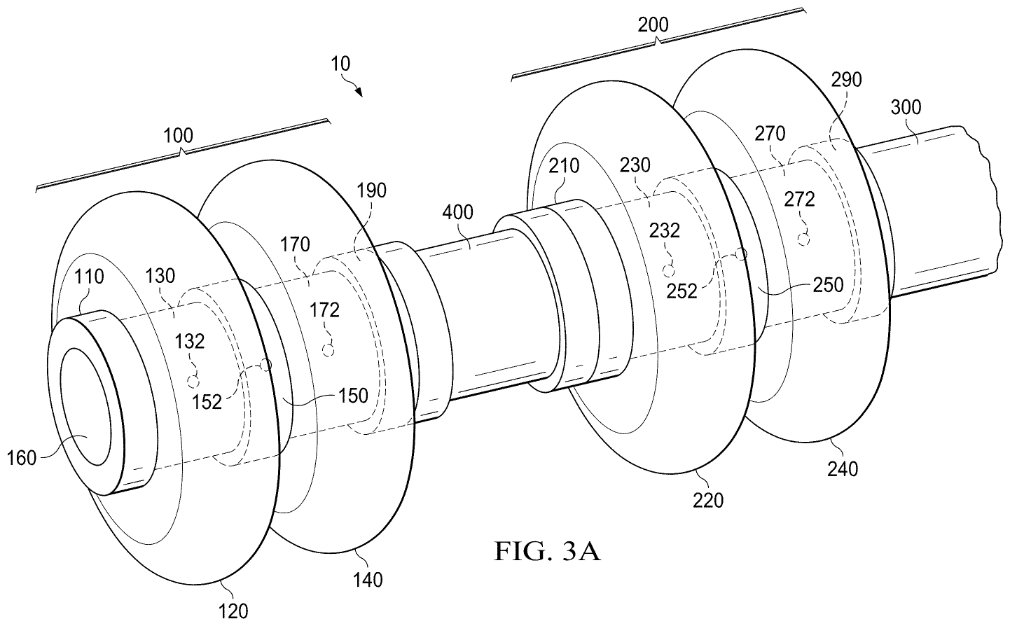

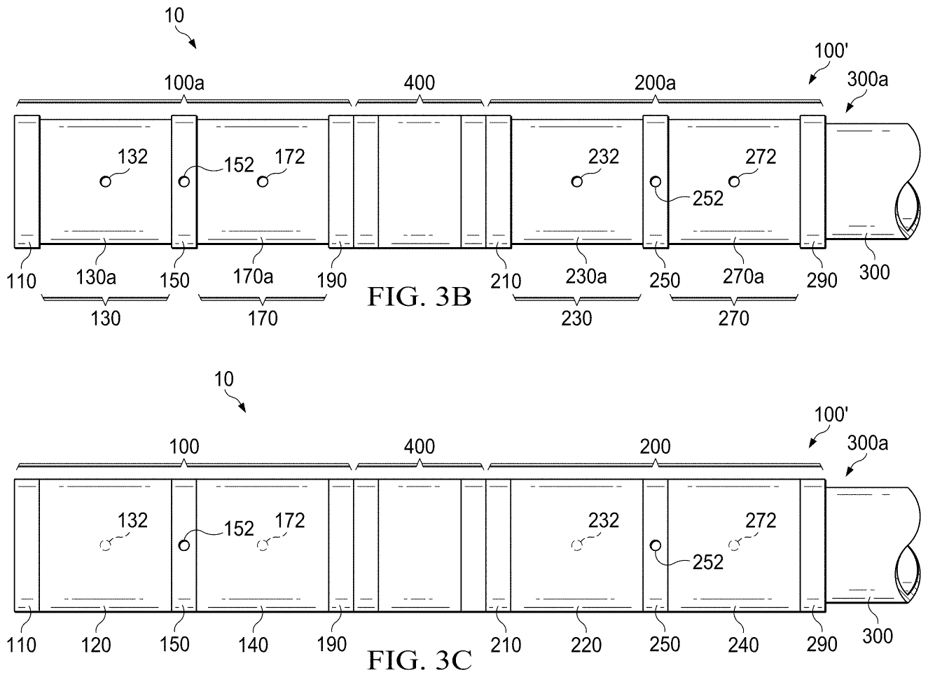

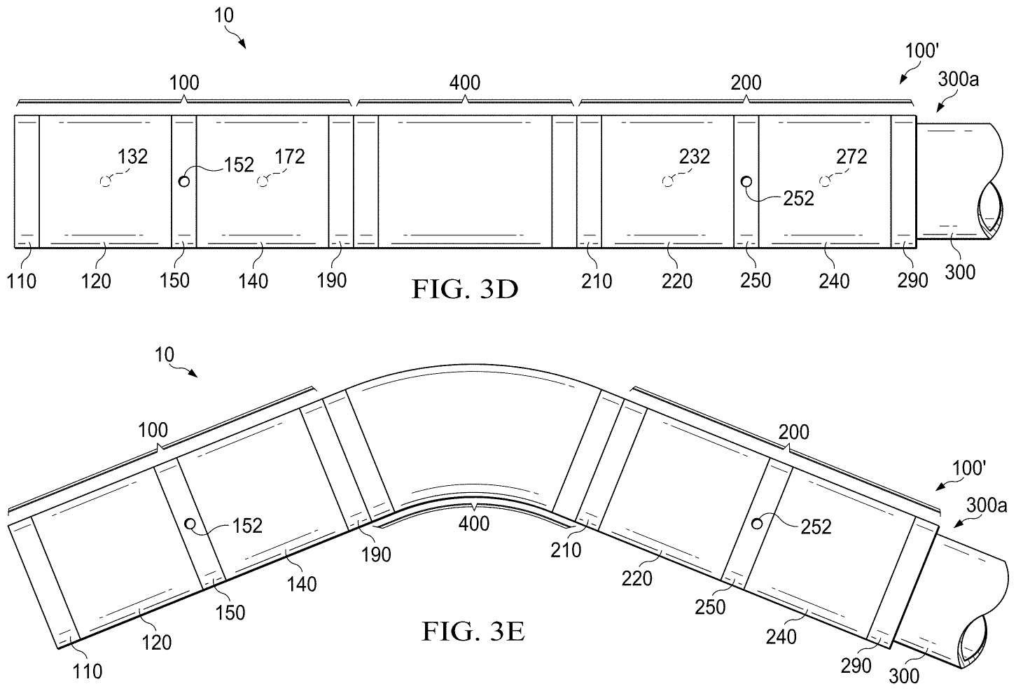

FIG. 1A is an illustration of a perspective view of an example embodiment of an endoscopic system (e.g., endoscopic system 10), FIG. 1B is an illustration of a side view of an example embodiment of an endoscopic system 10 without expandable members (e.g., expandable members 120, 140) secured to the main assembly body (e.g., main assembly body 100a illustrated in at least FIG. 1B), and FIG. 1C is an illustration of a side view of an example embodiment of an endoscopic system 10. FIG. 2A is an illustration of a perspective view of an example embodiment of an endoscopic system 10 with expandable members 120, 140 expanded outwardly away from the main assembly body 100a; and FIG. 2B is an illustration of a side view of an example embodiment of an endoscopic system 10 with the expandable members 120, 140 expanded outwardly away from the main assembly body 100a. FIG. 3A is an illustration of a perspective view of another example embodiment of an endoscopic system 10 with expandable members 120, 140 and expandable members 220, 240 expanded outwardly away from the main assembly body (e.g., first main assembly body 100a illustrated in at least FIG. 3C and second main assembly body 200a illustrated in at least FIG. 3C, respectively); FIG. 3B is an illustration of a side view of an example embodiment of an endoscopic system 10 without expandable members 120, 140 and expandable members 220, 240; and FIG. 3C is an illustration of a side view of an example embodiment of an endoscopic system 10 with expandable members 120, 140 and expandable members 220, 240 not expanded outwardly away from the main assembly body 100a, 200a, respectively.

An example embodiment of the endoscopic system 10 may include a main assembly (e.g., main assembly 100 illustrated in at least FIGS. 1A, 1C, 2A, and 2B and/or main assembly 200 illustrated in at least FIGS. 3A, 3C, 3D, and 3E). The endoscopic system 10 may also include an elongated tubular section (e.g., elongated tubular section 300 illustrated in at least FIGS. 1A-C, 2A-B, and 3A-E). A distal end (e.g., distal end 300a) of the elongated tubular section 300 may be secured to a proximal end (e.g., proximal end 100′) of the main assembly 100. A proximal end (not shown) of the elongated tubular section 300 may be connected to one or more surgeon control panels (not shown), one or more pressure sources (not shown), etc. The one or more pressure sources (not shown) may be used for providing positive pressure and/or negative pressure to one or more of the pressure openings (e.g., pressure openings 132, 152, 172 illustrated in at least FIGS. 1A-C and 2A-B) of the main assembly 100. The surgeon control panel (not shown) may include one or more computing devices, graphical displays, 3-D goggles/glasses, or the like, for one or more of the following: receiving and/or processing signals from an imaging assembly (e.g., a digital still and/or video camera; a 3-D stereoscopic or autostereoscopic device; etc.) at a most distal end (e.g., most distal end 160 as illustrated in at least FIGS. 1A, 2A, and 3A) of the main assembly 100; displaying images, including still pictures and/or video; controlling one or more actions or operations of the main assembly 100, 200 and/or other elements or functionality of the endoscopic system 10; etc. The most distal end 160 of the main assembly 100 may include other devices, elements, and/or functionality including, but not limited to, an illumination source for providing illumination (e.g., when inside a cavity of a patient); a suction and/or irrigation assembly (e.g., for removing liquids and/or solids from inside a cavity of a patient, and possibly for providing liquid and/or pressure into a cavity of a patient); a pressure opening (e.g., for providing positive pressure such as insufflation and/or negative pressure); one or more surgical instruments (e.g., for performing a surgical action); one or more sensors (e.g., for measuring temperature, humidity, etc.); etc.

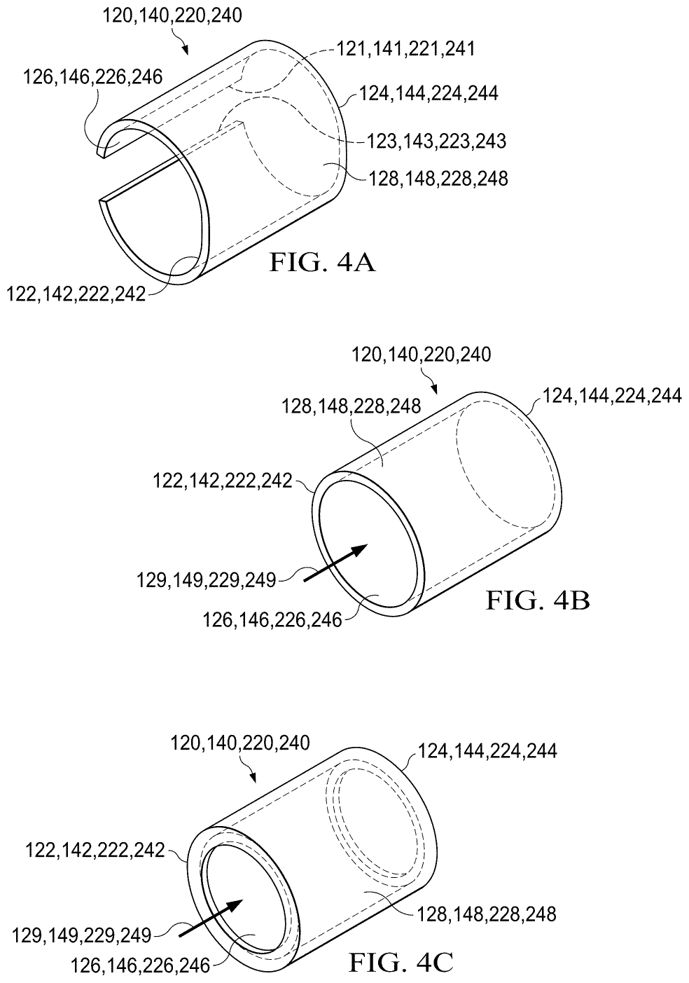

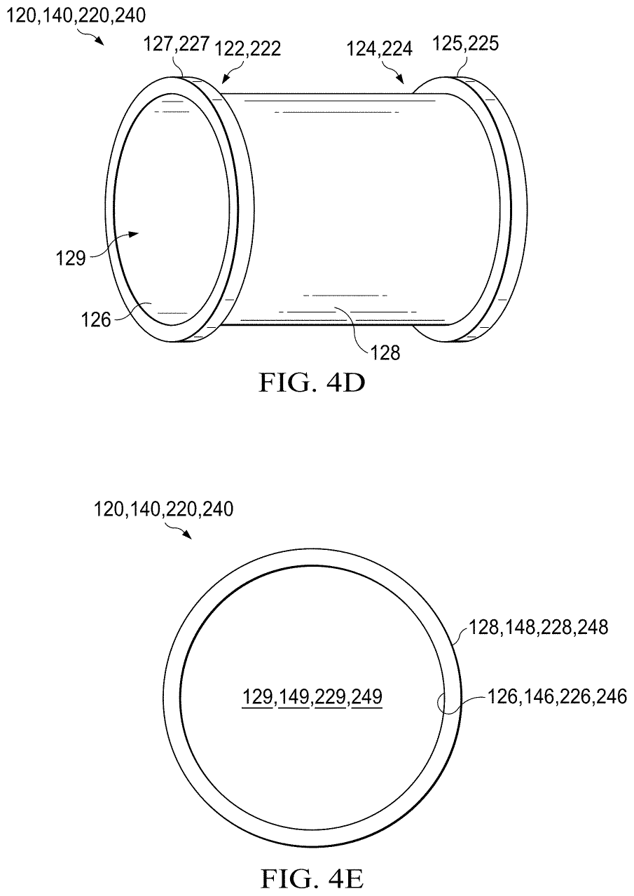

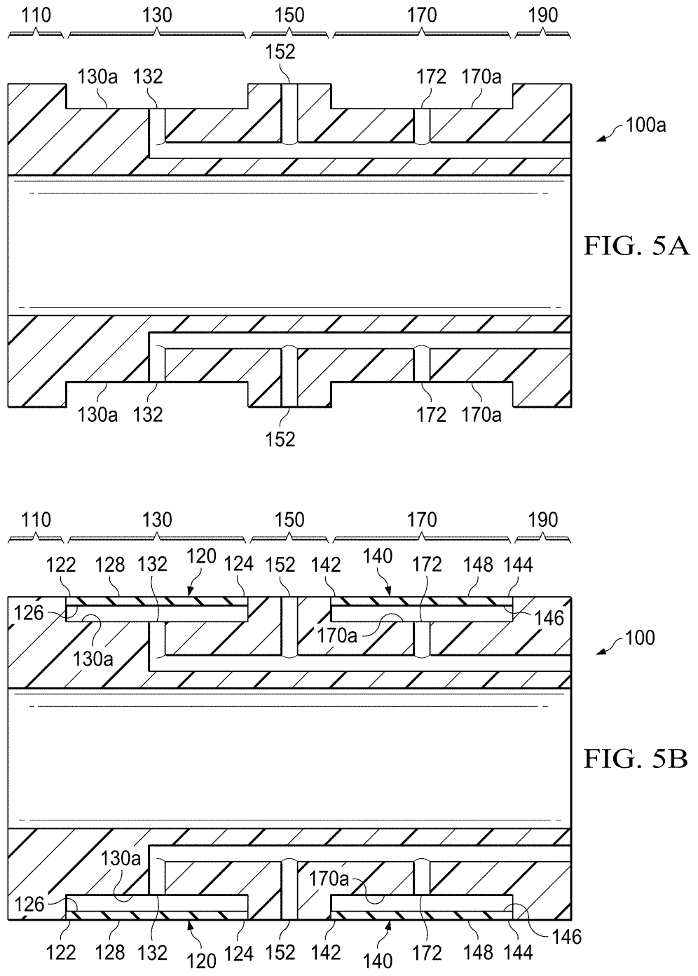

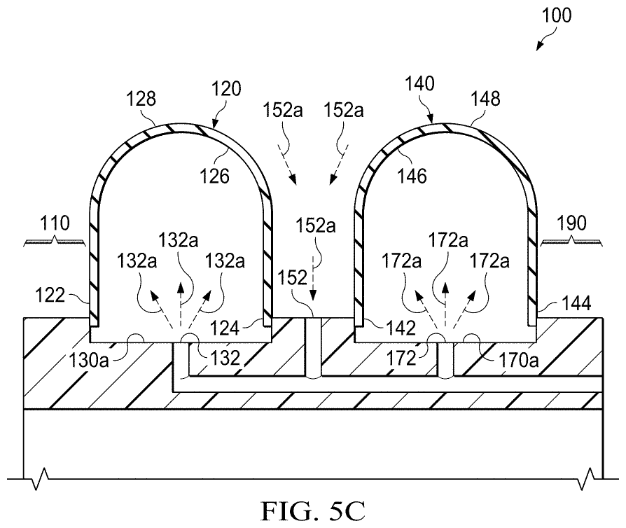

In terms of the main assembly 100, 200, the main assembly 100 may include a main assembly body (e.g., main assembly body 100a illustrated in at least FIGS. 1B and 3B and main assembly body 200a illustrated in at least FIG. 3B). The main assembly 100, 200 may also include one or more expandable members (e.g., expandable members 120, 140 illustrated in at least FIGS. 1A, 1C, 2A, 2B, 3A, and 3C-E; expandable members 220, 240 illustrated in at least FIGS. 3A and 3C-E; and expandable members 120, 140, 220, 240 illustrated in at least FIGS. 4A-D). The main assembly 100, 200 may also include one or more pressure openings (e.g., one or more pressure openings 132 illustrated in at least FIGS. 1A-C and 2A-B; one or more pressure openings 152 illustrated in at least FIGS. 1A-C and 2A-B; one or more pressure openings 172 illustrated in at least FIGS. 1A-C and 2A-B; one or more pressure openings 232 illustrated in at least FIGS. 3A-D; one or more pressure openings 252 illustrated in at least FIGS. 3A-D; one or more pressure openings 272 illustrated in at least FIGS. 3A-D). When inserted into a body cavity (e.g., the colon), the main assembly 100, 200 may be selectively configurable to anchor (or secure) the endoscopic system 10 to the cavity walls (e.g., colonic walls). As described in the present disclosure, such anchoring (or securing) may include one or more of the following: configuring one or more pressure openings 132, 172, 232, 272 to provide an outward positive pressure (e.g., outward positive pressure 132a, 172a illustrated in at least FIGS. 5C, 6C, and 7C); configuring one or more of the expandable members 120, 140, 220, 240 to expand outwardly away from the main assembly body 100a, 200a (or in an expanded configuration, as illustrated in at least FIGS. 1A, 2A, 2B, 3A, 5C, 6C, and 7C); and configuring one or more of the pressure openings 152, 252 to apply a negative pressure (e.g., negative pressure 152a illustrated in at least FIGS. 5C, 6C, and 7C in the region formed by an expanded expandable member 120, an expanded expandable member 140, and the cavity walls (e.g., colonic walls)).

These and other elements of the endoscopic system 10 will now be further described with reference to FIGS. 1 to 7.

Endoscopic System with Main Assembly (e.g., Main Assembly 100).

As illustrated in at least FIGS. 1A, 1C, 2A, and 2B, an example embodiment of the endoscopic system 10 may include a main assembly (e.g., main assembly 100) having expandable members (e.g., expandable member 120 and expandable member 140). FIG. 1D is an illustration of a perspective view of another example embodiment of the main assembly 100 having one expandable member (e.g., expandable member 120). The main assembly 100 may have an elongated structure, and/or may be cylindrical and/or tubular in shape. Although some of the figures may illustrate the main assembly 100 having an elongated cylindrical shape with a circular cross section, it is to be understood in the present disclosure that the main assembly 100 may be formed in any one or more other structures, shapes, and/or configurations. For example, a cross-section of the main assembly 100 may be in any one or more other shapes including, but not limited to, an oval, square, rectangle, hexagon, octagon, decagon, etc.

An example embodiment of the main assembly 100 may include a main assembly body (e.g., main assembly body 100a). The main assembly 100 may also include an expandable member (e.g., expandable member 120). The main assembly 100 may also include another expandable member (e.g., expandable member 140). The main assembly 100 may also include one or more pressure openings (e.g., pressure opening 132) for controlling or configuring the expandable member 120. The main assembly 100 may also include one or more other pressure openings (e.g., pressure opening 172) for controlling or configuring the expandable member 140. The main assembly 100 may also include one or more other pressure openings (e.g., pressure opening 152) for providing an outward positive pressure and/or negative pressure. The main assembly 100 may also include a most distal end (e.g., most distal end 160). These and other elements and functionality of the main assembly 100 will now be further described with reference to FIGS. 1 to 7.

(1) The Main Assembly Body (e.g., Main Assembly Body 100a).

The main assembly 100 may include a main assembly body (e.g., main assembly body 100a). The main assembly body 100a may be formed in one or more of a plurality of shapes, structures, and cross-sections. Although some of the figures may illustrate the main assembly body 100a to have an elongated cylindrical shape with a circular cross section, it is to be understood in the present disclosure that the main assembly body 100a may be formed in any one or more other structures, shapes, and/or configurations. For example, a cross-section of the main assembly body 100a may be in any one or more other shapes including, but not limited to, an oval, square, rectangle, hexagon, octagon, decagon, etc. An example embodiment of the main assembly body 100a is illustrated in at least FIG. 1B. FIG. 1E is an illustration of a side view of another example embodiment of the main assembly body 100a. Example embodiments of the main assembly 100 having the main assembly body 100a will now be further described with reference to the figures.

### Example Embodiment of the Main Assembly 100 Having Main Assembly Body 100a and Expandable Member 120 (as Illustrated in FIGS. 1D-E)

In the example embodiment illustrated in FIGS. 1D and 1E, the main assembly body 100a may include a proximal region (e.g., proximal region 190). The proximal region 190 may be a region, part, and/or portion of the main assembly body 100a. The proximal region 190 may have a same or similar cross-section as one or more other regions of the main assembly body 100a. The proximal region 190 may include an exterior surface. The proximal region 190 may correspond to the proximal end 100′ of the main assembly 100 that connects or secures to the distal end 300a of the elongated tubular member 300. The proximal region 190 may include one or more pressure openings (not shown). The one or more pressure openings of the proximal region 190 may be connected to or in communication with an external pressure source (not shown) via an internal channel or tube (not shown) in the elongated tubular member 300. The one or more pressure openings of the proximal region 190 may be configurable to provide a negative pressure (see for example the negative pressure 152a illustrated in FIGS. 5C, 6C, and 7C). It is recognized in the present disclosure that such negative pressure by the one or more pressure openings of the proximal region 190 (and/or pressure openings in the distal region 110 (not shown)) to “pull in” a portion of the cavity wall (e.g., colonic wall) in combination with the use of one or more expanded expandable members 120 and/or 140 may provide for a stronger anchoring (or securing) of the main assembly 100 to a cavity wall (e.g., colonic wall). The one or more pressure openings of the proximal region 190 may also be configurable to provide an outward positive pressure.

In an example embodiment, the proximal region 190 may have a length between about 1 mm to about 7 mm, and a diameter between about 8 mm to about 20 mm. Other dimensions and shapes are also contemplated without departing from the teachings of the present disclosure.

The main assembly body 100a may also include a distal region (e.g., distal region 110). The distal region 110 may be a region, part, and/or portion of the main assembly body 100a. The distal region 110 may have a same or similar cross-section as one or more other regions of the main assembly body 100a. The distal region 110 may include an exterior surface. The distal region 110 may include the most distal end 160. The distal region 110 may include one or more pressure openings (not shown). The one or more pressure openings of the distal region 110 may be connected to or in communication with an external pressure source (not shown) via an internal channel or tube (not shown) in the elongated tubular member 300. The one or more pressure openings of the distal region 110 may be configurable to provide a negative pressure (see for example the negative pressure 152a illustrated in FIGS. 5C, 6C, and 7C). It is recognized in the present disclosure that such negative pressure by the one or more pressure openings of the distal region 110 (and/or pressure openings in the proximal region 190 (not shown)) to “pull in” a portion of the cavity wall (e.g., colonic wall) in combination with the use of one or more expanded expandable members 120 and/or 140 may provide for a stronger anchoring (or securing) of the main assembly 100 to a cavity wall (e.g., colonic wall). The one or more pressure openings of the distal region 110 may also be configurable to provide an outward positive pressure.

In an example embodiment, the distal region 110 may have a length between about 1 mm to about 9 mm, and a diameter between about 8 mm to about 20 mm. Other dimensions and shapes are also contemplated without departing from the teachings of the present disclosure.

The main assembly body 100a may also include a central region (e.g., central region 130). The central region 130 may be a region of the main assembly body 100a between the proximal region 190 and distal region 110. The central region 130 may include an exterior surface 130a. The central region 130 may include one or more pressure openings (e.g., pressure opening 132). The one or more pressure openings 132 may be connected to or in communication with an external pressure source (not shown) via an internal channel or tube (not shown) in the elongated tubular member 300. The one or more pressure openings 132 may be configurable to provide an outward positive pressure (see for example the outward positive pressure 132a illustrated in FIGS. 5C, 6C, and 7C). The outward positive pressure provided by the one or more pressure openings 132 may exceed a threshold value required to cause the expandable member 120 to expand outwardly away from the exterior surface 130a of the central region 130. The one or more pressure openings 132 may also be configurable to provide a negative pressure, provide no pressure, or allow positive pressure to be released from the hermetically sealed cavity formed between the expandable member 120 and the exterior surface 130a of the central region 130 (e.g., to bring the expandable member 120 back to the normal or non-expanded state or configuration after providing an outward positive pressure to outwardly expand the expandable member 120 away from the central region 130). As illustrated in FIG. 1D and as will be further described in the present disclosure, the central region 130 may be received or covered by the expandable member 120. In this regard, when the expandable member 120 houses and/or receives the central region 130, an interior surface 126 (as also illustrated in at least FIGS. 4B-D) of the expandable member 120 faces (and in some embodiments, contacts with) the exterior surface 130a of the central region 130 of the main assembly body 100a. Furthermore, a first end 122 of the expandable member 120 may be secured to a portion of the distal region 110. Furthermore, a second end 124 of the expandable member 120 may be secured to a portion of the proximal region 190. As will be further described in the present disclosure, such securing of the first end 122 of the expandable member 120 to a portion of the distal region 110 and/or securing of the second end 124 of the expandable member 120 to a portion of the proximal region 190 may be performed in one or more of a plurality of ways. For example, the securing may be achieved via an overmolding process. As another example, the securing may be achieved via an adhesive.

In an example embodiment, the central region 130 may have a length between about 5 mm to about 15 mm, and a diameter between about 8 mm to about 20 mm. Other dimensions and shapes are also contemplated without departing from the teachings of the present disclosure.

### Example Embodiment of the Main Assembly 100 Having Main Assembly Body 100a and Expandable Members 120 and 140 (as Illustrated in FIG. 1A-C, 2, 3, and 5-7)

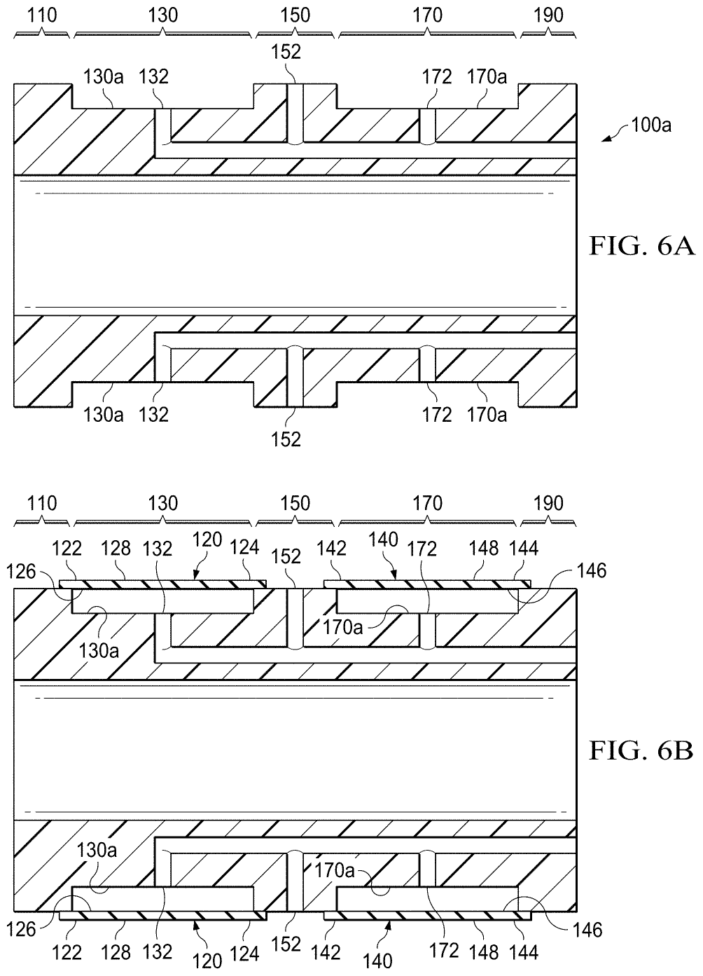

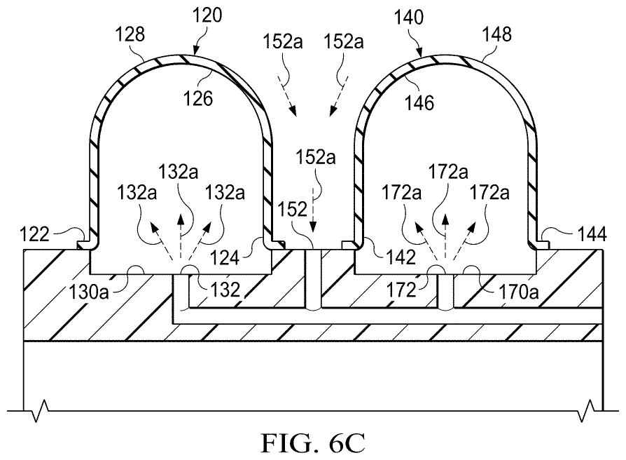

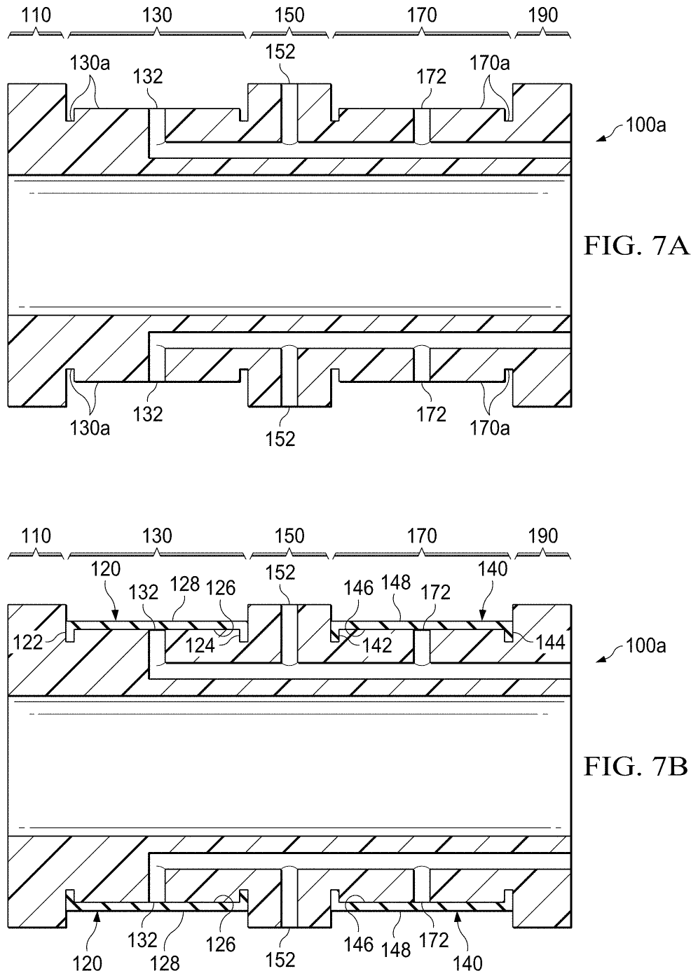

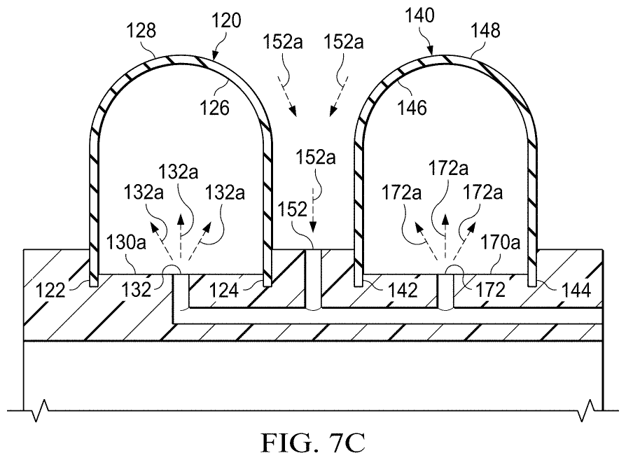

In the example embodiment illustrated in FIGS. 1A-C, 2A-B, and 3A-E and FIG. 5A, FIG. 5B, FIG. 5C, FIG. 6A, FIG. 6B, FIG. 6C, FIG. 7A, FIG. 7B, and FIG. 7C, the main assembly body 100a may include a proximal region (e.g., proximal region 190). The proximal region 190 may be a region, part, and/or portion of the main assembly body 100a. The proximal region 190 may have a same or similar cross-section as one or more other regions of the main assembly body 100a. The proximal region 190 may include an exterior surface. The proximal region 190 may correspond to the proximal end 100′ of the main assembly 100 that connects or secures to the distal end 300a of the elongated tubular member 300. proximal region 190 may include one or more pressure openings (not shown). The one or more pressure openings of the proximal region 190 may be connected to or in communication with an external pressure source (not shown) via an internal channel or tube (not shown) in the elongated tubular member 300. The one or more pressure openings of the proximal region 190 may be configurable to provide a negative pressure (see for example the negative pressure 152a illustrated in FIGS. 5C, 6C, and 7C). It is recognized in the present disclosure that such negative pressure by the one or more pressure openings of the proximal region 190 (and/or other pressure openings such as the one or more pressure openings 152 and/or pressure openings in the distal region 110 (not shown)) to “pull in” a portion of the cavity wall (e.g., colonic wall) in combination with the use of one or more expanded expandable members 120 and/or 140 may provide for a stronger anchoring (or securing) of the main assembly 100 to a cavity wall (e.g., colonic wall). The one or more pressure openings of the proximal region 190 may also be configurable to provide an outward positive pressure.

In an example embodiment, the proximal region 190 may have a length between about 1 mm to about 7 mm, and a diameter between about 8 mm to about 20 mm. Other dimensions and shapes are also contemplated without departing from the teachings of the present disclosure.

The main assembly body 100a may also include an intermediate region (e.g., intermediate region 150). The intermediate region 150 may be a region, part, and/or portion of the main assembly body 100a. The intermediate region 150 may have a same or similar cross-section as one or more other regions of the main assembly body 100a. The intermediate region 150 may include an exterior surface. The intermediate region 150 may include one or more pressure openings (e.g., pressure opening 152). The one or more pressure openings 152 may be connected to or in communication with an external pressure source (not shown) via an internal channel or tube (not shown) in the elongated tubular member 300. The one or more pressure openings 152 may be configurable to provide a negative pressure (see for example the negative pressure 152a illustrated in FIGS. 5C, 6C, and 7C). It is recognized in the present disclosure that such negative pressure by the one or more pressure openings 152 (and/or other pressure openings such as pressure openings in the proximal region 190 (not shown) and/or pressure openings in the distal region 110 (not shown)) to “pull in” a portion of the cavity wall (e.g., colonic wall) in combination with the use of one or more expanded expandable members 120 and/or 140 may provide for a stronger anchoring (or securing) of the main assembly 100 to a cavity wall (e.g., colonic wall). The one or more pressure openings 152 may also be configurable to provide an outward positive pressure.

In an example embodiment, the intermediate region 150 may have a length between about 1 mm to about 7 mm, and a diameter between about 8 mm to about 20 mm. Other dimensions and shapes are also contemplated without departing from the teachings of the present disclosure.

The main assembly body 100a may also include a first central region (e.g., central region 170). The first central region 170 may be a region of the main assembly body 100a between the proximal region 190 and intermediate region 150. The first central region 170 may include an exterior surface 170a. The first central region 170 may include one or more pressure openings (e.g., pressure opening 172). The one or more pressure openings 172 may be connected to or in communication with an external pressure source (not shown) via an internal channel or tube (not shown) in the elongated tubular member 300. The one or more pressure openings 172 may be configurable to provide an outward positive pressure (see for example the outward positive pressure 172a illustrated in FIGS. 5C, 6C, and 7C). The outward positive pressure provided by the one or more pressure openings 172 may exceed a threshold value required to cause the expandable member 140 to expand outwardly away from the exterior surface 170a of the first central region 170. The one or more pressure openings 172 may also be configurable to provide a negative pressure, provide no pressure, or allow positive pressure to be released from the hermetically sealed cavity formed between the expandable member 140 and the exterior surface 170a of the first central region 170 (e.g., to bring the expandable member 140 back to the normal or non-expanded state or configuration after providing an outward positive pressure to outwardly expand the expandable member 140 away from the first central region 170). As illustrated in FIGS. 1A, 1C, 2A, 2B, 3B-E, 5B, 5C, 6B, 6C, 7B, and 7C and as will be further described in the present disclosure, the first central region 170 may be received or covered by the expandable member 140. In this regard, when the expandable member 140 houses and/or receives the first central region 170, an interior surface 146 (as also illustrated in at least FIGS. 5B, 5C, 6B, 6C, 7B, and 7C) of the expandable member 140 faces (and in some embodiments, contacts with) the exterior surface 170a of the first central region 170 of the main assembly body 100a. Furthermore, as illustrated in at least FIGS. 5B, 5C, 6B, 6C, 7B, and 7C, a first end 142 of the expandable member 140 may be secured to a portion of the intermediate region 150; and a second end 144 of the expandable member 140 may be secured to a portion of the proximal region 190. As will be further described in the present disclosure, such securing of the first end 142 of the expandable member 140 to a portion of the intermediate region 150 and/or securing of the second end 144 of the expandable member 140 to a portion of the proximal region 190 may be performed in one or more of a plurality of ways. For example, the securing may be achieved via an overmolding process. As another example, the securing may be achieved via an adhesive.

In an example embodiment, the first central region 170 may have a length between about 5 mm to about 15 mm, and a diameter between about 8 mm to about 20 mm. Other dimensions and shapes are also contemplated without departing from the teachings of the present disclosure.

The main assembly body 100a may also include a distal region (e.g., distal region 110). The distal region 110 may be a region, part, and/or portion of the main assembly body 100a. The distal region 110 may have a same or similar cross-section as one or more other regions of the main assembly body 100a. The distal region 110 may include an exterior surface. The distal region 110 may also include the most distal end 160. The distal region 110 may include one or more pressure openings (not shown). The one or more pressure openings of the distal region 110 may be connected to or in communication with an external pressure source (not shown) via an internal channel or tube (not shown) in the elongated tubular member 300. The one or more pressure openings of the distal region 110 may be configurable to provide a negative pressure (see for example the negative pressure 152a illustrated in FIGS. 5C, 6C, and 7C). It is recognized in the present disclosure that such negative pressure by the one or more pressure openings of the distal region 110 (and/or other pressure openings such as pressure openings in the proximal region 190 (not shown) to “pull in” a portion of the cavity wall (e.g., colonic wall) in combination with the use of one or more expanded expandable members 120 and/or 140 may provide for a stronger anchoring (or securing) of the main assembly 100 to a cavity wall (e.g., colonic wall). The one or more pressure openings of the distal region 110 may also be configurable to provide an outward positive pressure.

In an example embodiment, the distal region 110 may have a length between about 1 mm to about 9 mm, and a diameter between about 8 mm to about 20 mm. Other dimensions and shapes are also contemplated without departing from the teachings of the present disclosure.

The main assembly body 100a may also include a second central region (e.g., central region 130). The second central region 130 may be a region of the main assembly body 100a between the intermediate region 150 and distal region 110. The second central region 130 may include an exterior surface 130a. The second central region 130 may include one or more pressure openings (e.g., pressure opening 132). The one or more pressure openings 132 may be connected to or in communication with an external pressure source (not shown) via an internal channel or tube (not shown) in the elongated tubular member 300. The one or more pressure openings 132 may be configurable to provide an outward positive pressure (see for example the outward positive pressure 132a illustrated in FIGS. 5C, 6C, and 7C). The outward positive pressure provided by the one or more pressure openings 132 may exceed a threshold value required to cause the expandable member 120 to expand outwardly away from the exterior surface 130a of the second central region 130. The one or more pressure openings 132 may also be configurable to provide a negative pressure, provide no pressure, or allow positive pressure to be released from the hermetically sealed cavity formed between the expandable member 120 and the exterior surface 130a of the second central region 130 (e.g., to bring the expandable member 120 back to the normal or non-expanded state or configuration after providing an outward positive pressure to outwardly expand the expandable member 120 away from the second central region 130). As illustrated in FIGS. 1A, 1C, 2A, 2B, 3B-E, 5B, 5C, 6B, 6C, 7B, and 7C and as will be further described in the present disclosure, the second central region 130 may be received or covered by the expandable member 120. In this regard, when the expandable member 120 houses and/or receives the second central region 130, an interior surface 126 (as also illustrated in at least FIGS. 4B-D, 5B, 5C, 6B, 6C, 7B, and 7C) of the expandable member 120 faces (and in some embodiments, contacts with) the exterior surface 130a of the second central region 130 of the main assembly body 100a. Furthermore, as illustrated in at least FIGS. 5B, 5C, 6B, 6C, 7B, and 7C, a first end 122 of the expandable member 120 may be secured to a portion of the distal region 110; and a second end 124 of the expandable member 120 may be secured to a portion of the intermediate region 150. As will be further described in the present disclosure, such securing of the first end 122 of the expandable member 120 to a portion of the distal region 110 and/or securing of the second end 124 of the expandable member 120 to a portion of the intermediate region 150 may be performed in one or more of a plurality of ways. For example, the securing may be achieved via an overmolding process. As another example, the securing may be achieved via an adhesive.

In an example embodiment, the second central region 130 may have a length between about 5 mm to about 15 mm and a diameter between about 8 mm to about 20 mm. Other dimensions and shapes are also contemplated without departing from the teachings of the present disclosure.

(2) The First Expandable Member (e.g., Expandable Member 120).

The main assembly 100 may include an expandable member (e.g., expandable member 120). The expandable member 120 may be formed in one or more of a plurality of shapes, structures, and cross-sections. Although some of the figures may illustrate the expandable member 120 to have a tubular (or cylindrical) shape with a circular cross section, it is to be understood in the present disclosure that the expandable member 120 may be formed in any one or more other structures, shapes, and/or configurations. For example, the expandable member 120 may not be tubular in shape. As another example, a cross-section of the expandable member 120 may be in any one or more other shapes including, but not limited to, an oval, square, rectangle, hexagon, octagon, decagon, etc. The expandable member 120 may be formed from one or more of a plurality of materials and/or compositions. For example, the expandable member 120 may be formed using surgical grade silicon rubber, liquid silicone rubber, TPE, TPU, etc.

Rectangular Shaped Expandable Member 120

As illustrated in FIG. 4A, an example embodiment of the expandable member 120 may be formed in the shape of a rectangular sheet, or the like. The expandable member 120 may include a first end (e.g., first end 124) and a second end (e.g., second end 122) opposite to the first end 124. The expandable member 120 may also include a third end (e.g., third end 121) and a fourth end (e.g., fourth end 123) opposite to the third end 121. The expandable member 120 may also include an interior surface (e.g., interior surface 126). The expandable member 120 may also include an exterior surface (e.g., exterior surface 128) opposite to the interior surface 126. The expandable member 120 may have a length between about 5 mm to about 15 mm, a width between about 8 mm to about 20 mm, and a thickness between about 0.1 mm to about 1 mm.

The expandable member 120 illustrated in FIG. 4A may be secured to the main assembly body 100a in one or more of a plurality of ways. For example, as illustrated in at least FIGS. 5B, 6B, and 7B, the first end 124 of the expandable member 120 may be secured to a portion of the intermediate region 150 and the second end 122 of the expandable member 120 may be secured to a portion of the distal region 110. Furthermore, the third end 121 of the expandable member 120 may be secured to a portion of the second central region 130 (such securing not shown) and the fourth end 123 of the expandable member 120 may be secured to another portion of the second central region 130 (such securing not shown). When the expandable member 120 is secured to the main assembly body 100a, the interior surface 126 of the expandable member 120 faces the exterior surface 130a of the second central region 130 of the main assembly body 100a. Furthermore, the interior surface 126 of the expandable member 120 faces the one or more pressure openings 132. In some example embodiments, at least a portion of the interior surface 126 of the expandable member 120 is in contact with at least a portion of the exterior surface 130a of the second central region 130 of the main assembly body 100a.

The securing of the expandable member 120 to the main assembly body 100a (including the securing of the first end 124 to the intermediate region 150, the securing of the second end 122 to the distal region 110, the securing of the third end 121 to the second central region 130, and the securing of the fourth end 123 to the second central region 130) may be performed in a hermetically sealed manner. That is, the securing of the expandable member 120 to the main assembly body 100a (including the securing of the first end 124 to the intermediate region 150, the securing of the second end 122 to the distal region 110, the securing of the third end 121 to the second central region 130, and the securing of the fourth end 123 to the second central region 130) may be performed in such a way that the expandable member 120 (or interior surface 126 of the expandable member 120) and second central region 130 (or the exterior surface 130a of the second central region 130) form a hermetically sealed chamber or cavity (provided the one or more pressure openings 132 are also not allowing pressure to be released). When the expandable member 120 is secured to the main assembly body 100a in a hermetically sealable manner so as to form a hermetically sealed chamber or cavity, an application or providing of a sufficient outward positive pressure 132a from the one or more pressure openings 132 (e.g., positive pressure exceeding a threshold value, such threshold value being a minimum value required to cause the expandable member 120 to expand) enables the expandable member 120 between the first end 124, second end 122, third end 121, and fourth end 123 to expand outwardly away from the second central region 130 of the main assembly body 100a.

For the example embodiment of the main assembly 100 illustrated in FIGS. 1D-E, the first end 124 of the expandable member 120 may be secured to a portion of the proximal region 190 and the second end 122 of the expandable member 120 may be secured to a portion of the distal region 110. Furthermore, the third end 121 of the expandable member 120 may be secured to a portion of the central region 130 (such securing not shown) and the fourth end 123 of the expandable member 120 may be secured to another portion of the central region 130 (such securing not shown). When the expandable member 120 is secured to the main assembly body 100a, the interior surface 126 of the expandable member 120 faces the exterior surface 130a of the central region 130 of the main assembly body 100a. Furthermore, the interior surface 126 of the expandable member 120 faces the one or more pressure openings 132. In some example embodiments, at least a portion of the interior surface 126 of the expandable member 120 is in contact with at least a portion of the exterior surface 130a of the central region 130 of the main assembly body 100a.

The securing of the expandable member 120 to the main assembly body 100a of FIGS. 1D-E (including the securing of the first end 124 to the proximal region 190, the securing of the second end 122 to the distal region 110, the securing of the third end 121 to the central region 130, and the securing of the fourth end 123 to the central region 130) may be performed in a hermetically sealed manner. That is, the securing of the expandable member 120 to the main assembly body 100a (including the securing of the first end 124 to the proximal region 190, the securing of the second end 122 to the distal region 110, the securing of the third end 121 to the central region 130, and the securing of the fourth end 123 to the central region 130) may be performed in such a way that the expandable member 120 (or interior surface 126 of the expandable member 120) and central region 130 (or the exterior surface 130a of the central region 130) form a hermetically sealed chamber or cavity (provided the one or more pressure openings 132 are also not allowing pressure to be released). When the expandable member 120 is secured to the main assembly body 100a in a hermetically sealable manner so as to form a hermetically sealed chamber or cavity, an application or providing of a sufficient outward positive pressure 132a from the one or more pressure openings 132 (e.g., positive pressure exceeding a threshold value, such threshold value being a minimum value required to cause the expandable member 120 to expand) enables the expandable member 120 between the first end 124, second end 122, third end 121, and fourth end 123 to expand outwardly away from the second central region 130 of the main assembly body 100a.

Tubular Shaped Expandable Member 120

As illustrated in at least FIG. 4B, FIG. 4C, FIG. 4D, and FIG. 4E, another example embodiment of the expandable member 120 may be formed in the shape of a tubular (or cylindrical) structure, or the like. When formed in a tubular structure, the expandable member 120 may include an interior channel (e.g., interior channel 129, as illustrated in at least FIG. 4E). The tubular structure of the expandable member 120 may include a first end (e.g., first end 124) (corresponding to a tubular end section of the tubular structure) and a second end (e.g., second end 122) (corresponding to another tubular end section of the tubular structure) opposite to the first end 124. The expandable member 120 may also include an interior surface (e.g., interior surface 126). Such interior surface 126 can be considered as forming the interior channel 129. The expandable member 120 may also include an exterior surface (e.g., exterior surface 128) opposite to the interior surface 126. Such exterior surface 128 may be for use in contacting with a cavity wall (e.g., colonic wall) so as to anchor (or secure) the main assembly 100 to the cavity wall when the expandable member 120 is expanded outwardly away from the exterior surface 130a of the second central region 130. In some example embodiments, the first end 124 of the expandable member 120 may be secured to a ring-shaped member 125 and the second end 122 of the expandable member 120 may be secured to a ring-shaped member 127 (see, for example, FIG. 4D). The expandable member 120 may have a length between about 5 mm to about 15 mm, a diameter between about 8 mm to about 20 mm, and a thickness between about 0.1 mm to about 1 mm.

The expandable member 120 illustrated in FIGS. 4B-E may be secured to the main assembly body 100a in one or more of a plurality of ways. For example, the interior channel 129 of the expandable member 120 may receive or house the second central region 130 of the main assembly body 100a. In doing so, the one or more pressure openings 132 of the second central region 130 may face the inward facing interior surface 126 of the expandable member 120. As illustrated in at least the cross-sectional illustrations of FIGS. 5B, 6B, and 7B, the first end 124 of the expandable member 120 (e.g., the entire circumference of the tubular end of the first end 124) may be secured to a portion of the intermediate region 150 and the second end 122 of the expandable member 120 (e.g., the entire circumference of the tubular end of the second end 122) may be secured to a portion of the distal region 110. In embodiments where the first end 124 of the expandable member 120 is secured to a ring-shaped member 125 and the second end 122 of the expandable member 120 is secured to a ring-shaped member 127 (see, for example, FIG. 4D), the securing of the expandable member 120 to the main assembly body 100a may include securing the ring-shaped member 125 to a portion of the intermediate region 150 and securing the ring-shaped member 127 to a portion of the distal region 110.

When the expandable member 120 is secured to the main assembly body 100a, the interior surface 126 of the expandable member 120 faces the exterior surface 130a of the second central region 130 of the main assembly body 100a. Furthermore, the interior surface 126 of the expandable member 120 faces the one or more pressure openings 132. In some example embodiments, at least a portion of the interior surface 126 of the expandable member 120 is in contact with at least a portion of the exterior surface 130a of the second central region 130 of the main assembly body 100a (see, for example, FIG. 7B).

The securing of the expandable member 120 to the main assembly body 100a (including the securing of the first end 124 to the intermediate region 150 and the securing of the second end 122 to the distal region 110) may be performed in a hermetically sealed manner. That is, the securing of the expandable member 120 to the main assembly body 100a (including the securing of the first end 124 to the intermediate region 150 and the securing of the second end 122 to the distal region 110) may be performed in such a way that the expandable member 120 (or interior surface 126 of the expandable member 120) and second central region 130 (or the exterior surface 130a of the second central region 130) form a hermetically sealed chamber or cavity (provided the one or more pressure openings 132 are also not allowing pressure to be released). As illustrated in at least FIGS. 5C, 6C, and 7C, when the expandable member 120 is secured to the main assembly body 100a in a hermetically sealable manner so as to form a hermetically sealed chamber or cavity, an application or providing of a sufficient outward positive pressure 132a from the one or more pressure openings 132 (e.g., positive pressure exceeding a threshold value, such threshold value being a minimum value required to cause the expandable member 120 to expand) enables the tubular structure of the expandable member 120 between the first end 124 and the second end 122 to expand outwardly away from the second central region 130 of the main assembly body 100a.

For the example embodiment of the main assembly 100 illustrated in FIGS. 1D-E, the interior channel 129 of the expandable member 120 may receive or house the central region 130 of the main assembly body 100a. In doing so, the one or more pressure openings 132 of the central region 130 may face the inward facing interior surface 126 of the expandable member 120. As illustrated in at least the cross-sectional illustrations of FIGS. 5B, 6B, and 7B, the first end 124 of the expandable member 120 (e.g., the entire circumference of the tubular end of the first end 124) may be secured to a portion of the proximal region 190 and the second end 122 of the expandable member 120 (e.g., the entire circumference of the tubular end of the second end 122) may be secured to a portion of the distal region 110. In embodiments where the first end 124 of the expandable member 120 is secured to a ring-shaped member 125 and the second end 122 of the expandable member 120 is secured to a ring-shaped member 127 (see, for example, FIG. 4D), the securing of the expandable member 120 to the main assembly body 100a may include securing the ring-shaped member 125 to a portion of the proximal region 190 and securing the ring-shaped member 127 to a portion of the distal region 110.

When the expandable member 120 is secured to the main assembly body 100a, the interior surface 126 of the expandable member 120 faces the exterior surface 130a of the central region 130 of the main assembly body 100a. Furthermore, the interior surface 126 of the expandable member 120 faces the one or more pressure openings 132. In some example embodiments, at least a portion of the interior surface 126 of the expandable member 120 is in contact with at least a portion of the exterior surface 130a of the central region 130 of the main assembly body 100a.

The securing of the expandable member 120 to the main assembly body 100a of FIGS. 1D-E (including the securing of the first end 124 to the proximal region 190 and the securing of the second end 122 to the distal region 110) may be performed in a hermetically sealed manner. That is, the securing of the expandable member 120 to the main assembly body 100a (including the securing of the first end 124 to the proximal region 190 and the securing of the second end 122 to the distal region 110) may be performed in such a way that the expandable member 120 (or interior surface 126 of the expandable member 120) and central region 130 (or the exterior surface 130a of the central region 130) form a hermetically sealed chamber or cavity (provided the one or more pressure openings 132 are also not allowing pressure to be released). As illustrated in at least FIGS. 5C, 6C, and 7C, when the expandable member 120 is secured to the main assembly body 100a in a hermetically sealable manner so as to form a hermetically sealed chamber or cavity, an application or providing of a sufficient outward positive pressure 132a from the one or more pressure openings 132 (e.g., positive pressure exceeding a threshold value, such threshold value being a minimum value required to cause the expandable member 120 to expand) enables the tubular structure of the expandable member 120 between the first end 124 and the second end 122 to expand outwardly away from the central region 130 of the main assembly body 100a.

(3) The Second Expandable Member (e.g., Expandable Member 140).

The main assembly 100 may include an expandable member (e.g., expandable member 140). The expandable member 140 may be formed in one or more of a plurality of shapes, structures, and cross-sections. Although some of the figures may illustrate the expandable member 140 to have a tubular (or cylindrical) shape with a circular cross section, it is to be understood in the present disclosure that the expandable member 140 may be formed in any one or more other structures, shapes, and/or configurations. For example, the expandable member 140 may not be tubular in shape. As another example, a cross-section of the expandable member 140 may be in any one or more other shapes including, but not limited to, an oval, square, rectangle, hexagon, octagon, decagon, etc. The expandable member 140 may be formed from one or more of a plurality of materials and/or compositions. For example, the expandable member 140 may be formed using surgical grade silicon rubber, liquid silicone rubber, TPE, TPU, etc.

Rectangular Shaped Expandable Member 140

Similar to the illustration in FIG. 4A, an example embodiment of the expandable member 140 may be formed in the shape of a rectangular sheet, or the like. The expandable member 140 may include a first end (e.g., first end 144) and a second end (e.g., second end 142) opposite to the first end 144. The expandable member 140 may also include a third end (e.g., third end 141) and a fourth end (e.g., fourth end 143) opposite to the third end 141. The expandable member 140 may also include an interior surface (e.g., interior surface 146). The expandable member 140 may also include an exterior surface (e.g., exterior surface 148) opposite to the interior surface 146. The expandable member 140 may have a length between about 5 mm to about 15 mm, a width between about 8 mm to about 20 mm, and a thickness between about 0.1 mm to about 1 mm.

The expandable member 140 may be secured to the main assembly body 100a in one or more of a plurality of ways. For example, as illustrated in at least FIGS. 5B, 6B, and 7B, the first end 144 of the expandable member 140 may be secured to a portion of the proximal region 190 and the second end 142 of the expandable member 140 may be secured to a portion of the intermediate region 150. Furthermore, the third end 141 of the expandable member 140 may be secured to a portion of the first central region 170 (such securing not shown) and the fourth end 143 of the expandable member 140 may be secured to another portion of the first central region 170 (such securing not shown). When the expandable member 140 is secured to the main assembly body 100a, the interior surface 146 of the expandable member 140 faces the exterior surface 170a of the first central region 170 of the main assembly body 100a. Furthermore, the interior surface 146 of the expandable member 140 faces the one or more pressure openings 172. In some example embodiments, at least a portion of the interior surface 146 of the expandable member 140 is in contact with at least a portion of the exterior surface 170a of the first central region 170 of the main assembly body 100a.

The securing of the expandable member 140 to the main assembly body 100a (including the securing of the first end 144 to the proximal region 190, the securing of the second end 142 to the intermediate region 150, the securing of the third end 141 to the first central region 170, and the securing of the fourth end 143 to the first central region 170) may be performed in a hermetically sealed manner. That is, the securing of the expandable member 140 to the main assembly body 100a (including the securing of the first end 144 to the proximal region 190, the securing of the second end 142 to the intermediate region 150, the securing of the third end 141 to the first central region 170, and the securing of the fourth end 143 to the first central region 170) may be performed in such a way that the expandable member 140 (or interior surface 146 of the expandable member 140) and first central region 170 (or the exterior surface 170a of the first central region 170) form a hermetically sealed chamber or cavity (provided the one or more pressure openings 172 are also not allowing pressure to be released). When the expandable member 140 is secured to the main assembly body 100a in a hermetically sealable manner so as to form a hermetically sealed chamber or cavity, an application or providing of a sufficient outward positive pressure 172a from the one or more pressure openings 172 (e.g., positive pressure exceeding a threshold value, such threshold value being a minimum value required to cause the expandable member 140 to expand) enables the expandable member 140 between the first end 144, second end 142, third end 141, and fourth end 143 to expand outwardly away from the first central region 170 of the main assembly body 100a.

Tubular Shaped Expandable Member 140

As illustrated in at least FIGS. 4B-E, another example embodiment of the expandable member 140 may be formed in the shape of a tubular (or cylindrical) structure, or the like. When formed in a tubular structure, the expandable member 140 may include an interior channel (e.g., interior channel 149, as illustrated in at least FIG. 4E). The tubular structure of the expandable member 140 may include a first end (e.g., first end 144) (corresponding to a tubular end section of the tubular structure) and a second end (e.g., second end 142) (corresponding to another tubular end section of the tubular structure) opposite to the first end 144. The expandable member 140 may also include an interior surface (e.g., interior surface 146). Such interior surface 146 can be considered as forming the interior channel 149. The expandable member 140 may also include an exterior surface (e.g., exterior surface 148) opposite to the interior surface 146. Such exterior surface 148 may be for use in contacting with a cavity wall (e.g., colonic wall) so as to anchor (or secure) the main assembly 100 to the cavity wall when the expandable member 140 is expanded outwardly away from the exterior surface 170a of the first central region 170. In some example embodiments, the first end 144 of the expandable member 140 may be secured to a ring-shaped member (similar to the ring-shaped member 125 in FIG. 4D) and the second end 142 of the expandable member 140 may be secured to a ring-shaped member (similar to the ring-shaped member 127 in FIG. 4D). The expandable member 140 may have a length between about 5 mm to about 15 mm, a diameter between about 8 mm to about 20 mm, and a thickness between about 0.1 mm to about 1 mm.

The expandable member 140 illustrated in FIGS. 4B-E may be secured to the main assembly body 100a in one or more of a plurality of ways. For example, the interior channel 149 of the expandable member 140 may receive or house the first central region 170 of the main assembly body 100a. In doing so, the one or more pressure openings 172 of the first central region 170 may face the inward facing interior surface 146 of the expandable member 140. As illustrated in at least the cross-sectional illustrations of FIGS. 5B, 6B, and 7B, the first end 144 of the expandable member 140 (e.g., the entire circumference of the tubular end of the first end 144) may be secured to a portion of the proximal region 190 and the second end 142 of the expandable member 140 (e.g., the entire circumference of the tubular end of the second end 142) may be secured to a portion of the intermediate region 150. In embodiments where the first end 144 of the expandable member 140 is secured to a ring-shaped member and the second end 142 of the expandable member 140 is secured to a ring-shaped member (see, for example, FIG. 4D), the securing of the expandable member 140 to the main assembly body 100a may include securing the ring-shaped member (that is secured to the first end 144) to a portion of the proximal region 190 and securing the ring-shaped member (that is secured to the second end 142) to a portion of the intermediate region 150.

When the expandable member 140 is secured to the main assembly body 100a, the interior surface 146 of the expandable member 140 faces the exterior surface 170a of the first central region 170 of the main assembly body 100a. Furthermore, the interior surface 146 of the expandable member 140 faces the one or more pressure openings 172. In some example embodiments, at least a portion of the interior surface 146 of the expandable member 140 is in contact with at least a portion of the exterior surface 170a of the first central region 170 of the main assembly body 100a (see, for example, FIG. 7B).

The securing of the expandable member 140 to the main assembly body 100a (including the securing of the first end 144 to the proximal region 190 and the securing of the second end 142 to the intermediate region 150) may be performed in a hermetically sealed manner. That is, the securing of the expandable member 140 to the main assembly body 100a (including the securing of the first end 144 to the proximal region 190 and the securing of the second end 142 to the intermediate region 150) may be performed in such a way that the expandable member 140 (or interior surface 146 of the expandable member 140) and first central region 170 (or the exterior surface 170a of the first central region 170) form a hermetically sealed chamber or cavity (provided the one or more pressure openings 172 are also not allowing pressure to be released). As illustrated in at least FIGS. 5C, 6C, and 7C, when the expandable member 140 is secured to the main assembly body 100a in a hermetically sealable manner so as to form a hermetically sealed chamber or cavity, an application or providing of a sufficient outward positive pressure 172a from the one or more pressure openings 172 (e.g., positive pressure exceeding a threshold value, such threshold value being a minimum value required to cause the expandable member 140 to expand) enables the tubular structure of the expandable member 140 between the first end 144 and the second end 142 to expand outwardly away from the first central region 170 of the main assembly body 100a.

(4) The Most Distal End (e.g., Most Distal End 160).

In an example embodiment, the main assembly 100 may include a most distal end (e.g., most distal end 160). The most distal end 160 may include other devices, elements, and/or functionality. For example, the most distal end 160 may include an illumination source for providing illumination (e.g., when inside a cavity of a patient). The most distal end 160 may also include a suction and/or irrigation assembly (e.g., for removing liquids and/or solids from inside a cavity of a patient, and possibly for providing liquid and/or pressure into a cavity of a patient). The most distal end 160 may also include a pressure opening (e.g., for providing positive pressure such as insufflation and/or negative pressure). The most distal end 160 may also include one or more surgical instruments (e.g., for performing a surgical action). The most distal end 160 may also include one or more sensors (e.g., for measuring temperature, humidity, etc.).

The elements of the most distal end 160, including those described above and in the present disclosure, may be in communication with a controller (not shown) via wired or wireless communications. In example embodiments where the communication between one or more of the elements of the most distal end 160 is via wires, such wiring may be provided or run through the main assembly body 100a and the elongated tubular member 300.

The Elongated Tubular Member (e.g., Elongated Tubular Member 300).

An example embodiment of the endoscopic system 10 may include an elongated tubular member (e.g., elongated tubular member 300). The elongated tubular member 300 may be a flexible tubular body having one or more internal channels. For example, the one or more internal channels may be provided to enable negative pressure and/or positive pressure to be supplied from one or more external pressure sources (not shown) to the one or more pressure openings 132, 152, 172. As another example, the one or more internal channels may be provided for positive pressure and/or negative pressure to be supplied from one or more external pressure sources (not shown) to the most distal end 160. As another example, the one or more internal channels may be provided for electrical and/or data cables to extend to the image capturing assembly in the most distal end 160. As another example, the one or more internal channels may be provided for one or more instruments (e.g., cutter, gripper, etc., not shown) to be provided through the most distal end 160 to perform a surgical action. In yet another example, the one or more internal channels may be provided for cables to extend to one or more sensors present in the most distal end 160 (e.g., for haptic feedback, temperature sensor, etc., not shown), etc. Other internal channels for other purposes are also contemplated in the present disclosure. It is to be understood that an internal channel of the elongated tubular member 300 may be any channel of the elongated tubular member 300 (including those that are wholly or partially within the elongated tubular member 300), and may include a smaller tube, or the like, provided in a larger channel or tube. It is also to be understood that an internal channel of the elongated tubular member 300 may extend beyond the proximal end 300a and/or distal end (not shown) of the elongated tubular member 300.

The distal end 300a of the elongated tubular member 300 may be securable to (and in example embodiments, detachable from) a proximal end 100a of the main assembly 100.

In an example embodiment, the elongated tubular member 300 may have a length between about 800 mm to about 2500 mm, and a diameter between about 10 mm to about 22 mm. The elongated tubular member 300 may be formed having one or more of a plurality of cross-sectional shapes, including a circular cross-section, elliptical cross-section, etc. Other dimensions and shapes are also contemplated without departing from the teachings of the present disclosure.

The Endoscopic System (e.g., the Endoscopic System 10 Illustrated in at Least FIG. 3) Having First and Second Main Assemblies (e.g., Main Assembly 100 and 200).

As illustrated in at least FIGS. 3A and C-E, an example embodiment of the endoscopic system 10 may include a first main assembly 100 and second main assembly (e.g., main assembly 200). Each of the first main assembly 100 and second main assembly 200 may have one or more expandable members (e.g., expandable member 120 and/or expandable member 140 for the first main assembly 100 and expandable member 220 and/or expandable member 240 for the second main assembly 200). The main assemblies 100 and/or 200 may have an elongated structure, and/or may be cylindrical and/or tubular in shape. Although some of the figures may illustrate the main assemblies 100 and 200 having an elongated cylindrical shape with a circular cross section, it is to be understood in the present disclosure that the main assemblies 100 and/or 200 may be formed in any one or more other structures, shapes, and/or configurations. For example, a cross-section of the main assemblies 100 and/or may be in any one or more other shapes including, but not limited to, an oval, square, rectangle, hexagon, octagon, decagon, etc. The endoscopic system 10 may also include an extendible section 400 between the first main assembly 100 and second main assembly 200. A distal end of the extendible section 400 may be secured to the proximal end of the first main assembly 100 and a proximal end of the extendible section 400 may be secured to the distal end of the second main assembly 200. In an example embodiment, the extendible section may include an extendible section body. The extendible section body may include a first extendible section end secured to the proximal end of the first main assembly 100. The extendible section body may also include a second extendible section end secured to the distal end of the second main assembly 200. The extendible section 400 may be configurable to adjust an overall length of the extendible section body between the first extendible section end and the second extendible section end. In an example embodiment, a distance between the proximal end of the first main assembly 100 and the distal end of the second main assembly 200 may be adjusted or changed by configuring the extendible section to adjust or change an overall length of the extendible section body. For example, as illustrated in at least FIG. 3C, the extendible section 400 may have a first overall length (and/or a first distance between the proximal end of the first main assembly 100 and the distal end of the second main assembly 200). As illustrated in at least FIG. 3D, the extendible section 400 may be configurable to adjust the overall length of the extendible section body between the first extendible section end and second extendible section end from the first overall length to a second overall length greater than the first overall length (and/or adjust the distance between the proximal end of the first main assembly 100 and the distal end of the second main assembly 200 from the first distance to a second distance greater than the first distance). The extendible section 400 (and/or another section, not shown) may be configurable to bend in one or more of a plurality of directions, curvatures, and/or sections, as illustrated in at least FIG. 3E.

The first and second main assemblies 100, 200 will now be further described with reference to FIGS. 1-7.

The First Main Assembly (e.g., the Main Assembly 100.

An example embodiment of the first main assembly 100 may include a first main assembly body (e.g., main assembly body 100a). The first main assembly 100 may also include an expandable member (e.g., expandable member 120). The first main assembly 100 may also include another expandable member (e.g., expandable member 140). The first main assembly 100 may also include one or more pressure openings (e.g., pressure opening 132) for controlling or configuring the expandable member 120. The first main assembly 100 may also include one or more other pressure openings (e.g., pressure opening 172) for controlling or configuring the expandable member 140. The first main assembly 100 may also include one or more other pressure openings (e.g., pressure opening 152) for providing an outward positive pressure and/or negative pressure. The first main assembly 100 may also include a most distal end (e.g., most distal end 160). These and other elements and functionality of the first main assembly 100 will now be further described with reference to FIGS. 1-7.

(1) The First Main Assembly Body (e.g., Main Assembly Body 100a).

The first main assembly 100 may include a first main assembly body (e.g., main assembly body 100a). The first main assembly body 100a may be similar to or substantially the same as the main assembly body 100 of FIGS. 1, 2, and 5-7, as described above and in the present disclosure.

(2) The First Expandable Member (e.g., Expandable Member 120).

The first main assembly 100 may include an expandable member (e.g., expandable member 120). The expandable member 120 may be similar to or substantially the same as the expandable member 120 of FIGS. 1, 2, and 4-7, as described above and in the present disclosure.

(3) The Second Expandable Member (e.g., Expandable Member 140).

The first main assembly 100 may include an expandable member (e.g., expandable member 140). The expandable member 140 may be similar to or substantially the same as the expandable member 140 of FIGS. 1, 2, and 4-7, as described above and in the present disclosure.

(4) The Most Distal End (e.g., Most Distal End 160).

In an example embodiment, the first main assembly 100 may include a most distal end (e.g., most distal end 160). The most distal end 160 may include other devices, elements, and/or functionality. The most distal end 160 may be similar to or substantially the same as the most distal end 160 of FIGS. 1 and 2, as described above and in the present disclosure.

The Second Main Assembly (e.g., Main Assembly 200).

An example embodiment of the second main assembly 200 may include a second main assembly body (e.g., main assembly body 200a). The second main assembly 200 may also include an expandable member (e.g., expandable member 220). The second main assembly 200 may also include another expandable member (e.g., expandable member 240). The second main assembly 200 may also include one or more pressure openings (e.g., pressure opening 232) for controlling or configuring the expandable member 220. The second main assembly 200 may also include one or more other pressure openings (e.g., pressure opening 272) for controlling or configuring the expandable member 240. The second main assembly 200 may also include one or more other pressure openings (e.g., pressure opening 252) for providing an outward positive pressure and/or negative pressure. These and other elements and functionality of the second main assembly 200 will now be further described with reference to FIGS. 1 to 7.

(1) The Second Main Assembly Body (e.g., Main Assembly Body 200a).

The second main assembly 200 may include a second main assembly body (e.g., main assembly body 200a). The second main assembly body 200a may be formed in one or more of a plurality of shapes, structures, and cross-sections. Although some of the figures may illustrate the second main assembly body 200a having an elongated cylindrical shape with a circular cross section, it is to be understood in the present disclosure that the second main assembly body 200a may be formed in any one or more other structures, shapes, and/or configurations. For example, a cross-section of the second main assembly body 200a may be in any one or more other shapes including, but not limited to, an oval, square, rectangle, hexagon, octagon, decagon, etc. An example embodiment of the second main assembly body 200a is illustrated in at least FIG. 3B. Example embodiments of the second main assembly 200 having the second main assembly body 200a will now be further described with reference to the figures.

### Example Embodiment of the Second Main Assembly 200 Having Second Main Assembly Body 200a and Expandable Members 220 and 240 (as Illustrated in at Least FIG. 3A-E)

In the example embodiment illustrated in FIGS. 3A-E, the second main assembly body 200a may include a proximal region (e.g., proximal region 290). The proximal region 290 may be a region, part, and/or portion of the second main assembly body 200a. The proximal region 290 may have a same or similar cross-section as one or more other regions of the second main assembly body 200a. The proximal region 290 may include an exterior surface. The proximal region 290 may correspond to the proximal end of the second main assembly 200 that connects or secures to the distal end 300a of the elongated tubular member 300. The proximal region 290 may include one or more pressure openings (not shown). The one or more pressure openings of the proximal region 290 may be connected to or in communication with an external pressure source (not shown) via an internal channel or tube (not shown) in the elongated tubular member 300. The one or more pressure openings of the proximal region 290 may be configurable to provide a negative pressure (similar to the negative pressure 152a illustrated in FIGS. 5C, 6C, and 7C for pressure openings 152). It is recognized in the present disclosure that such negative pressure by the one or more pressure openings of the proximal region 290 (and/or other pressure openings such as the one or more pressure openings 252 and/or pressure openings in the distal region 210 (not shown)) to “pull in” a portion of the cavity wall (e.g., colonic wall) in combination with the use of one or more expanded expandable members 220 and/or 240 may provide for a stronger anchoring (or securing) of the second main assembly 200 to a cavity wall (e.g., colonic wall). The one or more pressure openings of the proximal region 290 may also be configurable to provide an outward positive pressure.

In an example embodiment, the proximal region 290 may have a length between about 1 mm to about 7 mm, and a diameter between about 8 mm to about 20 mm. Other dimensions and shapes are also contemplated without departing from the teachings of the present disclosure.

The second main assembly body 200a may also include an intermediate region (e.g., intermediate region 250). The intermediate region 250 may be a region, part, and/or portion of the second main assembly body 200a. The intermediate region 250 may have a same or similar cross-section as one or more other regions of the second main assembly body 200a. The intermediate region 250 may include an exterior surface. The intermediate region 250 may include one or more pressure openings (e.g., pressure opening 252). The one or more pressure openings 252 may be connected to or in communication with an external pressure source (not shown) via an internal channel or tube (not shown) in the elongated tubular member 300. The one or more pressure openings 252 may be configurable to provide a negative pressure (similar to the negative pressure 152a illustrated in FIGS. 5C, 6C, and 7C for pressure openings 152). It is recognized in the present disclosure that such negative pressure by the one or more pressure openings 252 (and/or other pressure openings such as pressure openings in the proximal region 290 (not shown) and/or pressure openings in the distal region 210 (not shown)) to “pull in” a portion of the cavity wall (e.g., colonic wall) in combination with the use of one or more expanded expandable members 220 and/or 240 may provide for a stronger anchoring (or securing) of the second main assembly 200 to a cavity wall (e.g., colonic wall). The one or more pressure openings 252 may also be configurable to provide an outward positive pressure.

In an example embodiment, the intermediate region 250 may have a length between about 1 mm to about 7 mm, and a diameter between about 8 mm to about 20 mm. Other dimensions and shapes are also contemplated without departing from the teachings of the present disclosure.

The second main assembly body 200a may also include a first central region (e.g., central region 270). The first central region 270 may be a region of the second main assembly body 200a between the proximal region 290 and intermediate region 250. The first central region 270 may include an exterior surface 270a. The first central region 270 may include one or more pressure openings (e.g., pressure opening 272). The one or more pressure openings 272 may be connected to or in communication with an external pressure source (not shown) via an internal channel or tube (not shown) in the elongated tubular member 300. The one or more pressure openings 272 may be configurable to provide an outward positive pressure (see for example the outward positive pressure 172a illustrated in FIGS. 5C, 6C, and 7C). The outward positive pressure provided by the one or more pressure openings 272 may exceed a threshold value required to cause the expandable member 240 to expand outwardly away from the exterior surface 270a of the first central region 270. The one or more pressure openings 272 may also be configurable to provide a negative pressure, provide no pressure, or allow positive pressure to be released from the hermetically sealed cavity formed between the expandable member 240 and the exterior surface 270a of the first central region 270 (e.g., to bring the expandable member 240 back to the normal or non-expanded state or configuration after providing an outward positive pressure to outwardly expand the expandable member 240 away from the first central region 270). As illustrated in FIGS. 1A, 1C, 2A, 2B, 5B, 5C, 6B, 6C, 7B, and 7C for the main assembly 100 and FIGS. 3A-E for the first and second main assemblies 100, 200 and as will be further described in the present disclosure, the first central region 270 may be received or covered by the expandable member 240. In this regard, when the expandable member 240 houses and/or receives the first central region 270, an interior surface 246 (as also illustrated in at least FIGS. 5B, 5C, 6B, 6C, 7B, and 7C for expandable member 140) of the expandable member 240 faces (and in some embodiments, contacts with) the exterior surface 270a of the first central region 270 of the second main assembly body 200a. Furthermore, as illustrated in at least FIGS. 5B, 5C, 6B, 6C, 7B, and 7C for expandable member 140, a first end 242 of the expandable member 240 may be secured to a portion of the intermediate region 250; and a second end 244 of the expandable member 240 may be secured to a portion of the proximal region 290. As will be further described in the present disclosure, such securing of the first end 242 of the expandable member 240 to a portion of the intermediate region 250 and/or securing of the second end 244 of the expandable member 240 to a portion of the proximal region 290 may be performed in one or more of a plurality of ways. For example, the securing may be achieved via an overmolding process. As another example, the securing may be achieved via an adhesive.

In an example embodiment, the first central region 270 may have a length between about 5 mm to about 15 mm, and a diameter between about 8 mm to about 20 mm. Other dimensions and shapes are also contemplated without departing from the teachings of the present disclosure.

The second main assembly body 200a may also include a distal region (e.g., distal region 210). The distal region 210 may be a region, part, and/or portion of the main assembly body 200a. The distal region 210 may have a same or similar cross-section as one or more other regions of the second main assembly body 200a. The distal region 210 may include an exterior surface. The distal region 210 may include one or more pressure openings (not shown). The one or more pressure openings of the distal region 210 may be connected to or in communication with an external pressure source (not shown) via an internal channel or tube (not shown) in the elongated tubular member 300. The one or more pressure openings of the distal region 210 may be configurable to provide a negative pressure (see for example the negative pressure 152a illustrated in FIGS. 5C, 6C, and 7C for pressure openings 152). It is recognized in the present disclosure that such negative pressure by the one or more pressure openings of the distal region 210 (and/or other pressure openings such as pressure openings in the proximal region 290 (not shown) and/or pressure openings in the distal region 210 (not shown)) to “pull in” a portion of the cavity wall (e.g., colonic wall) in combination with the use of one or more expanded expandable members 220 and/or 240 may provide for a stronger anchoring (or securing) of the second main assembly 200 to a cavity wall (e.g., colonic wall). The one or more pressure openings of the distal region 210 may also be configurable to provide an outward positive pressure.

In an example embodiment, the distal region 210 may have a length between about 1 mm to about 9 mm, and a diameter between about 8 mm to about 20 mm. Other dimensions and shapes are also contemplated without departing from the teachings of the present disclosure.

The second main assembly body 200a may also include a second central region (e.g., central region 230). The second central region 230 may be a region of the second main assembly body 200a between the intermediate region 250 and distal region 210. The second central region 230 may include an exterior surface 230a. The second central region 230 may include one or more pressure openings (e.g., pressure opening 232). The one or more pressure openings 232 may be connected to or in communication with an external pressure source (not shown) via an internal channel or tube (not shown) in the elongated tubular member 300. The one or more pressure openings 232 may be configurable to provide an outward positive pressure (see for example the outward positive pressure 132a illustrated in FIGS. 5C, 6C, and 7C for pressure openings 132). The outward positive pressure provided by the one or more pressure openings 232 may exceed a threshold value required to cause the expandable member 220 to expand outwardly away from the exterior surface 230a of the second central region 230. The one or more pressure openings 232 may also be configurable to provide a negative pressure, provide no pressure, or allow positive pressure to be released from the hermetically sealed cavity formed between the expandable member 220 and the exterior surface 230a of the second central region 230 (e.g., to bring the expandable member 220 back to the normal or non-expanded state or configuration after providing an outward positive pressure to outwardly expand the expandable member 220 away from the second central region 230). As illustrated in FIGS. 1A, 1C, 2A, 2B, 5B, 5C, 6B, 6C, 7B, and 7C for the main assembly 100 and FIGS. 3A-E for the first and second main assemblies 100, 200 and as will be further described in the present disclosure, the second central region 230 may be received or covered by the expandable member 220. In this regard, when the expandable member 220 houses and/or receives the second central region 230, an interior surface 226 (as also illustrated in at least FIGS. 4B-D, 5B, 5C, 6B, 6C, 7B, and 7C for expandable member 120) of the expandable member 220 faces (and in some embodiments, contacts with) the exterior surface 230a of the second central region 230 of the second main assembly body 200a. Furthermore, as illustrated in at least FIGS. 5B, 5C, 6B, 6C, 7B, and 7C for the main assembly 100, a first end 222 of the expandable member 220 may be secured to a portion of the distal region 210; and a second end 224 of the expandable member 220 may be secured to a portion of the intermediate region 250. As will be further described in the present disclosure, such securing of the first end 222 of the expandable member 220 to a portion of the distal region 210 and/or securing of the second end 224 of the expandable member 220 to a portion of the intermediate region 250 may be performed in one or more of a plurality of ways. For example, the securing may be achieved via an overmolding process. As another example, the securing may be achieved via an adhesive.

In an example embodiment, the second central region 230 may have a length between about 5 mm to about 15 mm and a diameter between about 8 mm to about 20 mm. Other dimensions and shapes are also contemplated without departing from the teachings of the present disclosure.

(2) The First Expandable Member (e.g., Expandable Member 220).

The second main assembly 200 may include an expandable member (e.g., expandable member 220). The expandable member 220 may be formed in one or more of a plurality of shapes, structures, and cross-sections. Although some of the figures may illustrate the expandable member 220 to have a tubular (or cylindrical) shape with a circular cross section, it is to be understood in the present disclosure that the expandable member 220 may be formed in any one or more other structures, shapes, and/or configurations. For example, the expandable member 220 may not be tubular in shape. As another example, a cross-section of the expandable member 220 may be in any one or more other shapes including, but not limited to, an oval, square, rectangle, hexagon, octagon, decagon, etc. The expandable member 220 may be formed from one or more of a plurality of materials and/or compositions. For example, the expandable member 220 may be formed using surgical grade silicon rubber, liquid silicone rubber, TPE, TPU, etc.

As illustrated in at least FIGS. 4B-E for expandable member 120, an example embodiment of the expandable member 220 may be formed in the shape of a tubular (or cylindrical) structure, or the like. When formed in a tubular structure, the expandable member 220 may include an interior channel (e.g., interior channel 229, as illustrated in at least FIG. 4E). The tubular structure of the expandable member 220 may include a first end (e.g., first end 224) (corresponding to a tubular end section of the tubular structure) and a second end (e.g., second end 222) (corresponding to another tubular end section of the tubular structure) opposite to the first end 224. The expandable member 220 may also include an interior surface (e.g., interior surface 226). Such interior surface 226 can be considered as forming the interior channel 229. The expandable member 220 may also include an exterior surface (e.g., exterior surface 228) opposite to the interior surface 226. Such exterior surface 228 may be for use in contacting with a cavity wall (e.g., colonic wall) so as to anchor (or secure) the second main assembly 200 to the cavity wall when the expandable member 220 is expanded outwardly away from the exterior surface 230a of the second central region 230. In some example embodiments, the first end 224 of the expandable member 220 may be secured to a ring-shaped member 225 and the second end 222 of the expandable member 220 may be secured to a ring-shaped member 227 (see, for example, FIG. 4D). The expandable member 220 may have a length between about 5 mm to about 15 mm, a diameter between about 8 mm to about 20 mm, and a thickness between about 0.1 mm to about 1 mm.

The expandable member 220 illustrated in FIGS. 4B-E may be secured to the second main assembly body 200a in one or more of a plurality of ways. For example, the interior channel 229 of the expandable member 220 may receive or house the second central region 230 of the second main assembly body 200a. In doing so, the one or more pressure openings 232 of the second central region 230 may face the inward facing interior surface 226 of the expandable member 220. As illustrated in at least the cross-sectional illustrations of FIGS. 5B, 6B, and 7B for expandable member 120, the first end 224 of the expandable member 220 (e.g., the entire circumference of the tubular end of the first end 224) may be secured to a portion of the intermediate region 250 and the second end 222 of the expandable member 220 (e.g., the entire circumference of the tubular end of the second end 222) may be secured to a portion of the distal region 210. In embodiments where the first end 224 of the expandable member 220 is secured to a ring-shaped member 225 and the second end 222 of the expandable member 220 is secured to a ring-shaped member 227 (see, for example, FIG. 4D), the securing of the expandable member 220 to the main assembly body 200a may include securing the ring-shaped member 225 to a portion of the intermediate region 250 and securing the ring-shaped member 227 to a portion of the distal region 210.

When the expandable member 220 is secured to the second main assembly body 200a, the interior surface 226 of the expandable member 220 faces the exterior surface 230a of the second central region 230 of the second main assembly body 200a. Furthermore, the interior surface 226 of the expandable member 220 faces the one or more pressure openings 232. In some example embodiments, at least a portion of the interior surface 226 of the expandable member 220 is in contact with at least a portion of the exterior surface 230a of the second central region 230 of the second main assembly body 200a (see, for example, FIG. 7B).

The securing of the expandable member 220 to the second main assembly body 200a (including the securing of the first end 224 to the intermediate region 250 and the securing of the second end 222 to the distal region 210) may be performed in a hermetically sealed manner. That is, the securing of the expandable member 220 to the second main assembly body 200a (including the securing of the first end 224 to the intermediate region 250 and the securing of the second end 222 to the distal region 210) may be performed in such a way that the expandable member 220 (or interior surface 226 of the expandable member 220) and second central region 230 (or the exterior surface 230a of the second central region 230) form a hermetically sealed chamber or cavity (provided the one or more pressure openings 232 are also not allowing pressure to be released). As illustrated in at least FIGS. 5C, 6C, and 7C for expandable member 120, when the expandable member 220 is secured to the second main assembly body 200a in a hermetically sealable manner so as to form a hermetically sealed chamber or cavity, an application or providing of a sufficient outward positive pressure from the one or more pressure openings 232 (e.g., positive pressure exceeding a threshold value, such threshold value being a minimum value required to cause the expandable member 220 to expand) enables the tubular structure of the expandable member 220 between the first end 224 and the second end 222 to expand outwardly away from the second central region 230 of the second main assembly body 200a.

(3) The Second Expandable Member (e.g., Expandable Member 240).

The second main assembly 200 may include an expandable member (e.g., expandable member 240). The expandable member 240 may be formed in one or more of a plurality of shapes, structures, and cross-sections. Although some of the figures may illustrate the expandable member 240 to have a tubular (or cylindrical) shape with a circular cross section, it is to be understood in the present disclosure that the expandable member 240 may be formed in any one or more other structures, shapes, and/or configurations. For example, the expandable member 240 may not be tubular in shape. As another example, a cross-section of the expandable member 240 may be in any one or more other shapes including, but not limited to, an oval, square, rectangle, hexagon, octagon, decagon, etc. The expandable member 240 may be formed from one or more of a plurality of materials and/or compositions. For example, the expandable member 240 may be formed using surgical grade silicon rubber, liquid silicone rubber, TPE, TPU, etc.

As illustrated in at least FIGS. 4B-E, an example embodiment of the expandable member 240 may be formed in the shape of a tubular (or cylindrical) structure, or the like. When formed in a tubular structure, the expandable member 240 may include an interior channel (e.g., interior channel 249, as illustrated in at least FIG. 4E). The tubular structure of the expandable member 240 may include a first end (e.g., first end 244) (corresponding to a tubular end section of the tubular structure) and a second end (e.g., second end 242) (corresponding to another tubular end section of the tubular structure) opposite to the first end 244. The expandable member 240 may also include an interior surface (e.g., interior surface 246). Such interior surface 246 can be considered as forming the interior channel 249. The expandable member 240 may also include an exterior surface (e.g., exterior surface 248) opposite to the interior surface 246. Such exterior surface 248 may be for use in contacting with a cavity wall (e.g., colonic wall) so as to anchor (or secure) the second main assembly 200 to the cavity wall when the expandable member 240 is expanded outwardly away from the exterior surface 270a of the first central region 270. In some example embodiments, the first end 244 of the expandable member 240 may be secured to a ring-shaped member (similar to the ring-shaped member 125 in FIG. 4D) and the second end 242 of the expandable member 240 may be secured to a ring-shaped member (similar to the ring-shaped member 127 in FIG. 4D). The expandable member 240 may have a length between about 5 mm to about 15 mm, a diameter between about 8 mm to about 20 mm, and a thickness between about 0.1 mm to about 1 mm.

The expandable member 240 illustrated in FIGS. 4B-E may be secured to the second main assembly body 200a in one or more of a plurality of ways. For example, the interior channel 249 of the expandable member 240 may receive or house the first central region 270 of the second main assembly body 200a. In doing so, the one or more pressure openings 272 of the first central region 270 may face the inward facing interior surface 246 of the expandable member 240. As illustrated in at least the cross-sectional illustrations of FIGS. 5B, 6B, and 7B for expandable member 140, the first end 244 of the expandable member 240 (e.g., the entire circumference of the tubular end of the first end 244) may be secured to a portion of the proximal region 290 and the second end 242 of the expandable member 240 (e.g., the entire circumference of the tubular end of the second end 242) may be secured to a portion of the intermediate region 250. In embodiments where the first end 244 of the expandable member 240 is secured to a ring-shaped member and the second end 242 of the expandable member 240 is secured to a ring-shaped member (see, for example, FIG. 4D), the securing of the expandable member 240 to the second main assembly body 200a may include securing the ring-shaped member (that is secured to the first end 242) to a portion of the proximal region 290 and securing the ring-shaped member (that is secured to the second end 242) to a portion of the intermediate region 250.

When the expandable member 240 is secured to the second main assembly body 200a, the interior surface 246 of the expandable member 240 faces the exterior surface 270a of the first central region 270 of the second main assembly body 200a. Furthermore, the interior surface 246 of the expandable member 240 faces the one or more pressure openings 272. In some example embodiments, at least a portion of the interior surface 246 of the expandable member 240 is in contact with at least a portion of the exterior surface 270a of the first central region 270 of the second main assembly body 200a (see, for example, FIG. 7B).

The securing of the expandable member 240 to the second main assembly body 200a (including the securing of the first end 244 to the proximal region 290 and the securing of the second end 242 to the intermediate region 250) may be performed in a hermetically sealed manner. That is, the securing of the expandable member 240 to the second main assembly body 200a (including the securing of the first end 244 to the proximal region 290 and the securing of the second end 242 to the intermediate region 250) may be performed in such a way that the expandable member 240 (or interior surface 246 of the expandable member 240) and first central region 270 (or the exterior surface 270a of the first central region 270) form a hermetically sealed chamber or cavity (provided the one or more pressure openings 272 are also not allowing pressure to be released). As illustrated in at least FIGS. 5C, 6C, and 7C for expandable member 140, when the expandable member 240 is secured to the second main assembly body 200a in a hermetically sealable manner so as to form a hermetically sealed chamber or cavity, an application or providing of a sufficient outward positive pressure 272a from the one or more pressure openings 272 (e.g., positive pressure exceeding a threshold value, such threshold value being a minimum value required to cause the expandable member 240 to expand) enables the tubular structure of the expandable member 240 between the first end 244 and the second end 242 to expand outwardly away from the first central region 270 of the second main assembly body 200a.

The Elongated Tubular Member (e.g., Elongated Tubular Member 300).

An example embodiment of the endoscopic system 10 may include an elongated tubular member (e.g., elongated tubular member 300). The elongated tubular member 300 may be a flexible tubular body having one or more internal channels. For example, the one or more internal channels may be provided to enable negative pressure and/or positive pressure to be supplied from one or more external pressure sources (not shown) to the one or more pressure openings 132, 152, 172, 232, 252, 272. As another example, the one or more internal channels may be provided for positive pressure and/or negative pressure to be supplied from one or more external pressure sources (not shown) to the most distal end 160. As another example, the one or more internal channels may be provided for electrical and/or data cables to extend to the image capturing assembly in the most distal end 160. As another example, the one or more internal channels may be provided for one or more instruments (e.g., cutter, gripper, etc., not shown) to be provided through the most distal end 160 to perform a surgical action. In yet another example, the one or more internal channels may be provided for cables to extend to one or more sensors present in the most distal end 160 (e.g., for haptic feedback, temperature sensor, etc., not shown), etc. Other internal channels for other purposes are also contemplated in the present disclosure. It is to be understood that an internal channel of the elongated tubular member 300 may be any channel of the elongated tubular member 300 (including those that are wholly or partially within the elongated tubular member 300), and may include a smaller tube, or the like, provided in a larger channel or tube. It is also to be understood that an internal channel of the elongated tubular member 300 may extend beyond the proximal end 300a and/or distal end (not shown) of the elongated tubular member 300.

The distal end 300a of the elongated tubular member 300 may be securable to (and in example embodiments, detachable from) a proximal end 100a of the second main assembly 200.

In an example embodiment, the elongated tubular member 300 may have a length between about 800 mm to about 2500 mm, and a diameter between about 10 mm to about 22 mm. The elongated tubular member 300 may be formed having one or more of a plurality of cross-sectional shapes, including a circular cross-section, elliptical cross-section, etc. Other dimensions and shapes are also contemplated without departing from the teachings of the present disclosure.

While various embodiments in accordance with the disclosed principles have been described above, it should be understood that they have been presented by way of example only, and are not limiting. Thus, the breadth and scope of the example embodiments described in the present disclosure should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

For example, “assembly,” “device,” “portion,” “segment,” “member,” “body,” or other similar terms should generally be construed broadly to include one part or more than one part attached or connected together.

Various terms used herein have special meanings within the present technical field. Whether a particular term should be construed as such a “term of art” depends on the context in which that term is used. “Connected,” “connecting,” “attached,” “attaching,” “anchored,” “anchoring,” “in communication with,” “communicating with,” “associated with,” “associating with,” or other similar terms should generally be construed broadly to include situations where attachments, connections, and anchoring are direct between referenced elements or through one or more intermediaries between the referenced elements. These and other terms are to be construed in light of the context in which they are used in the present disclosure and as one of ordinary skill in the art would understand those terms in the disclosed context. The above definitions are not exclusive of other meanings that might be imparted to those terms based on the disclosed context.

As referred to in the present disclosure, a computing device, a processor, and/or a system may be a virtual machine, computer, node, instance, host, and/or device in a networked or non-networked computing environment. A networked computing environment may be a collection of devices connected by communication channels that facilitate communications between devices and allow devices to share resources. Also as referred to in the present disclosure, a computing device may be a device deployed to execute a program operating as a socket listener and may include software instances.

Resources may encompass any type of resource for running instances including hardware (such as servers, clients, mainframe computers, networks, network storage, data sources, memory, central processing unit time, scientific instruments, and other computing devices), as well as software, software licenses, available network services, and other non-hardware resources, or a combination thereof.

A networked computing environment may include, but is not limited to, computing grid systems, distributed computing environments, cloud computing environment, etc. Such networked computing environments include hardware and software infrastructures configured to form a virtual organization comprised of multiple resources that may be in geographically disperse locations.

Furthermore, the coverage of the present application and any patents issuing from the present application may extend to one or more communications protocols, including TCP/IP.

Words of comparison, measurement, and timing such as “at the time,” “equivalent,” “during,” “complete,” and the like should be understood to mean “substantially at the time,” “substantially equivalent,” “substantially during,” “substantially complete,” etc., where “substantially” means that such comparisons, measurements, and timings are practicable to accomplish the implicitly or expressly stated desired result.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, a description of a technology in the “Background” is not to be construed as an admission that technology is prior art to any invention(s) in this disclosure. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings herein.

What is claimed is:

1. An endoscopic system comprising:
a main assembly, the main assembly having:
a main assembly body, the main assembly body being cylindrical in shape and having a proximal region, a distal region, and a central region between the proximal and distal regions, the central region having one or more first pressure openings formed through an exterior surface of the central region, the one or more first pressure openings configurable to provide an outward positive pressure, wherein the proximal region, central region, and distal region are collectively formed in such a way as to have a common central axis; and
an expandable member secured to the main assembly body, the expandable member having a tubular structure with an interior channel, an inward facing interior surface forming the interior channel, an outward facing exterior surface opposite to the inward facing interior surface, a first end corresponding to an end of the tubular structure of the expandable member, and a second end corresponding to another end of the tubular structure of the expandable member, wherein:
the interior channel of the expandable member houses the central region of the main assembly body in such a way that the one or more first pressure openings face the inward facing interior surface of the expandable member;
the first end of the expandable member is secured to the proximal region of the main assembly body; and
the second end of the expandable member is secured to the distal region of the main assembly body;
wherein, when the one or more first pressure openings provide an outward positive pressure, the tubular structure of the expandable member between the first and second ends of the expandable member is configured to expand outwardly away from the central region of the main assembly body.

2. The endoscopic system of claim 1, wherein one or more of the following apply:
the first end of the expandable member is secured to the proximal region of the main assembly body in a hermetically sealed manner; and/or
the second end of the expandable member is secured to the distal region of the main assembly body in a hermetically sealed manner; and/or
the first end of the expandable member is secured to the proximal region of the main assembly body via an overmolding process; and/or
the second end of the expandable member is secured to the distal region of the main assembly body via an overmolding process.

3. The endoscopic system of claim 1, wherein one or more of the following apply:
the first end of the expandable member is secured to a first ring-shaped member, and the first end of the expandable member is secured to the proximal region of the main assembly body by securing the first ring-shaped member to the proximal region of the main assembly body; and/or
the second end of the expandable member is secured to a second ring-shaped member, and the second end of the expandable member is secured to the distal region of the main assembly body by securing the second ring-shaped member to the distal region of the main assembly body.

4. The endoscopic system of claim 1, wherein one or more of the following apply:
the common central axis of the proximal region, central region, and distal region corresponds to the central axis of the cylindrically shaped main assembly body; and/or
each of the central region, proximal region, and distal region are cylindrical in shape; and/or
the tubular structure of the expandable member is a single thin-walled tubular structure; and/or
the proximal region, central region, and distal region are formed as a unitary body.

5. The endoscopic system of claim 1, further comprising a pressure source connected to the one or more first pressure openings;
wherein, when the first and second ends of the expandable member are secured to the proximal and distal regions of the main assembly body, respectively, the pressure source and the one or more first pressure openings are collectively configurable to cooperate with the expandable member and the exterior surface of the central region of the main assembly body to form a hermetically sealed chamber.

6. The endoscopic system of claim 1, wherein:
when the expandable member is not expanded outwardly by the outward positive pressure of the one or more first pressure openings, at least a portion of the inward facing interior surface of the expandable member is in contact with at least a portion of the exterior surface of the central region of the main assembly body.

7. The endoscopic system of claim 1, wherein:
when the expandable member is not expanded outwardly by the outward positive pressure of the one or more first pressure openings, at least a portion of the inward facing interior surface of the expandable member is not in contact with the exterior surface of the central region of the main assembly body.

8. The endoscopic system of claim 1, wherein:
when the one or more first pressure openings provide an outward positive pressure exceeding a threshold value, the tubular structure of the expandable member between the first and second ends of the expandable member is configured to expand outwardly away from the exterior surface of the central region of the main assembly body in such a way as to form a hollow toroidal-shaped body surrounding the central region of the main assembly body.

9. The endoscopic system of claim 8, wherein:
the hollow toroidal-shaped body includes a substantially D-shaped cross-section with a flat inner side and a curved side, the flat inner side corresponding to the exterior surface of the central region of the main assembly body and the curved side corresponding at least a portion of the expandable member.

10. The endoscopic system of claim 1, further comprising an elongated tubular member having proximal and distal ends, the distal end of the elongated tubular member secured to the proximal region of the main assembly body.

11. The endoscopic system of claim 1, wherein the proximal region of the main assembly body includes one or more second pressure openings, the one or more second pressure openings configurable to provide an inward negative pressure.

12. An endoscopic system comprising:
a main assembly, the main assembly having:
a main assembly body, the main assembly body having:
a proximal region;
an intermediate region;
a first central region between the proximal and intermediate regions, the first central region having one or more first pressure openings formed through an exterior surface of the first central region;
a distal region; and
a second central region between the intermediate and distal regions, the second central region having one or more second pressure openings formed through an exterior surface of the second central region;
wherein the proximal region, first central region, intermediate region, second central region, and distal region are collectively formed in such a way as to have a common central axis;
a first expandable member secured to the main assembly body, the first expandable member having a tubular structure with a first interior channel, an inward facing first interior surface forming the first interior channel, an outward facing first exterior surface opposite to the inward facing first interior surface, a first end corresponding to an end of the tubular structure of the first expandable member, and a second end corresponding to another end of the tubular structure of the first expandable member, wherein:
the first interior channel of the first expandable member houses the first central region of the main assembly body in such a way that the one or more first pressure openings face the inward facing first interior surface of the first expandable member;
the first end of the first expandable member is secured to the proximal region of the main assembly body; and
the second end of the first expandable member is secured to the intermediate region of the main assembly body;
wherein, when the one or more first pressure openings provide an outward positive pressure, the tubular structure of the first expandable member between the first and second ends of the first expandable member is configured to expand outwardly away from the first central region of the main assembly body.

13. The endoscopic system of claim 12, wherein the main assembly further comprises:
a second expandable member secured to the main assembly body, the second expandable member having a tubular structure with a second interior channel, an inward facing second interior surface forming the second interior channel, an outward facing second exterior surface opposite to the inward facing second interior surface, a first end corresponding to an end of the tubular structure of the second expandable member, and a second end corresponding to another end of the tubular structure of the second expandable member.

14. The endoscopic system of claim 13, wherein:
the second interior channel of the second expandable member houses the second central region of the main assembly body in such a way that the one or more second pressure openings face the inward facing second interior surface of the second expandable member;
the first end of the second expandable member is secured to the intermediate region of the main assembly body; and
the second end of the second expandable member is secured to the distal region of the main assembly body.

15. The endoscopic system of claim 14, wherein:
when the one or more second pressure openings provide an outward positive pressure, the tubular structure of the second expandable member between the first and second ends of the second expandable member is configured to expand outwardly away from the second central region of the main assembly body.

16. The endoscopic system of claim 12, wherein:
when the one or more first pressure openings provide an outward positive pressure exceeding a threshold value, the tubular structure of the first expandable member between the first and second ends of the first expandable member is configured to expand outwardly away from the exterior surface of the first central region of the main assembly body in such a way as to form a hollow toroidal-shaped body surrounding the first central region of the main assembly body.

17. The endoscopic system of claim 16, wherein:
the hollow toroidal-shaped body includes a substantially D-shaped cross-section with a flat inner side and a curved side, the flat inner side corresponding to the exterior surface of the first central region of the main assembly body and the curved side corresponding at least a portion of the first expandable member.

18. The endoscopic system of claim 13, wherein:
when the one or more second pressure openings provide an outward positive pressure exceeding a threshold value, the tubular structure of the second expandable member between the first and second ends of the second expandable member is configured to expand outwardly away from the exterior surface of the second central region of the main assembly body in such a way as to form a hollow toroidal-shaped body surrounding the second central region of the main assembly body.

19. The endoscopic system of claim 18, wherein:
the hollow toroidal-shaped body includes a substantially D-shaped cross-section with a flat inner side and a curved side, the flat inner side corresponding to the exterior surface of the second central region of the main assembly body and the curved side corresponding at least a portion of the second expandable member.

20. The endoscopic system of claim 12, wherein the intermediate region of the main assembly body includes one or more third pressure openings, the one or more third pressure openings configurable to provide an inward negative pressure.

21. An endoscopic system comprising:
a first main assembly, the first main assembly having:
a first main assembly body, the first main assembly body having:
a first proximal region;
a first intermediate region;
a first proximal central region between the first proximal region and first intermediate region, the first proximal central region having one or more first pressure openings formed through an exterior surface of the first proximal central region;
a first distal region; and
a first distal central region between the first intermediate region and first distal region;
wherein the first proximal region, first proximal central region, first intermediate region, first distal central region, and first distal region are collectively formed in such a way as to have a common first central axis; and
a first expandable member secured to the first main assembly body, the first expandable member having a tubular structure with a first interior channel, an inward facing first interior surface forming the first interior channel, an outward facing first exterior surface opposite to the inward facing first interior surface, a first end corresponding to an end of the tubular structure of the first expandable member, and a second end corresponding to another end of the tubular structure of the first expandable member, wherein:
the first interior channel of the first expandable member houses the first proximal central region of the first main assembly body in such a way that the one or more first pressure openings face the inward facing first interior surface of the first expandable member;
the first end of the first expandable member is secured to the first proximal region of the first main assembly body; and
the second end of the first expandable member is secured to the first intermediate region of the first main assembly body;
wherein, when the one or more first pressure openings provide an outward positive pressure, the tubular structure of the first expandable member between the first and second ends of the first expandable member is configured to expand outwardly away from the first proximal central region of the first main assembly body; and
a second main assembly adjustably positionable relative to the first main assembly, the first and second main assemblies configured in such a way that a distance between the first and second main assemblies is adjustable, the second main assembly having:
a second main assembly body, the second main assembly body having:
a second proximal region;
a second intermediate region;
a second proximal central region between the second proximal region and second intermediate region, the second proximal central region having one or more second pressure openings formed through an exterior surface of the second proximal central region;
a second distal region; and
a second distal central region between the second intermediate region and second distal region;
wherein the second proximal region, second proximal central region, second intermediate region, second distal central region, and second distal region are collectively formed in such a way as to have a common second central axis; and
a second expandable member secured to the second main assembly body, the second expandable member having a tubular structure with a second interior channel, an inward facing second interior surface forming the second interior channel, an outward facing second exterior surface opposite to the inward facing second interior surface, a first end corresponding to an end of the tubular structure of the second expandable member, and a second end corresponding to another end of the tubular structure of the second expandable member, wherein:
the second interior channel of the second expandable member houses the second proximal central region of the second main assembly body in such a way that the one or more second pressure openings face the inward facing second interior surface of the second expandable member;
the first end of the second expandable member is secured to the second proximal region of the second main assembly body; and
the second end of the second expandable member is secured to the second intermediate region of the second main assembly body;
wherein, when the one or more second pressure openings provide an outward positive pressure, the tubular structure of the second expandable member between the first and second ends of the second expandable member is configured to expand outwardly away from the second proximal central region of the second main assembly body.

22. The endoscopic system of claim 21, wherein the first main assembly further comprises:
a third expandable member secured to the first main assembly body, the third expandable member having a tubular structure with a third interior channel, an inward facing third interior surface forming the third interior channel, an outward facing third exterior surface opposite to the inward facing third interior surface, a first end corresponding to an end of the tubular structure of the third expandable member, and a second end corresponding to another end of the tubular structure of the third expandable member.

23. The endoscopic system of claim 22, wherein:
the first distal central region of the first main assembly body includes one or more third pressure openings formed through an exterior surface of the first distal central region;
the third interior channel of the third expandable member houses the first distal central region of the first main assembly body in such a way that the one or more third pressure openings face the inward facing third interior surface of the third expandable member;
the first end of the third expandable member is secured to the first intermediate region of the first main assembly body; and
the second end of the third expandable member is secured to the first distal region of the first main assembly body.

24. The endoscopic system of claim 23, wherein:
when the one or more third pressure openings provide an outward positive pressure, the tubular structure of the third expandable member between the first and second ends of the third expandable member is configured to expand outwardly away from the first distal central region of the first main assembly body.

25. The endoscopic system of claim 21, wherein:
when the one or more first pressure openings provide an outward positive pressure exceeding a threshold value, the tubular structure of the first expandable member between the first and second ends of the first expandable member is configured to expand outwardly away from the exterior surface of the first proximal central region of the first main assembly body in such a way as to form a hollow toroidal-shaped body surrounding the first proximal central region of the main assembly body.

26. The endoscopic system of claim 21, wherein:
when the one or more second pressure openings provide an outward positive pressure exceeding a threshold value, the tubular structure of the second expandable member between the first and second ends of the second expandable member is configured to expand outwardly away from the exterior surface of the second proximal central region of the second main assembly body in such a way as to form a hollow toroidal-shaped body surrounding the second proximal central region of the second main assembly body.

27. The endoscopic system of claim 21, wherein one or more of the following apply:
the first distal region includes an image capturing assembly; and/or
the first intermediate region includes one or more fourth pressure openings, the one or more fourth pressure openings configurable to provide an inward negative pressure; and/or
the second intermediate region includes one or more fifth pressure openings, the one or more fifth pressure openings configurable to provide an inward negative pressure; and/or
the first end of the first expandable membrane sheet is secured to the first proximal region of the first main assembly body via an overmolding process; and/or
the second end of the first expandable membrane sheet is secured to the first intermediate region of the first main assembly body via an overmolding process.

28. The endoscopic system of claim 21, further comprising:
an extendible section, the extendible section having an extendible section body, the extendible section body having a first extendible section end secured to the first main assembly and a second extendible section end secured to the second main assembly, the extendible section configurable to adjust an overall length of the extendible section body between the first extendible section end and the second extendible section end;
wherein the distance between the first and second main assemblies is changed by configuring the extendible section to change the overall length of the extendible section body.

29. An endoscopic system comprising:
a main assembly, the main assembly having:
a main assembly body, the main assembly body being cylindrical in shape and having a proximal region, a distal region, and a central region between the proximal and distal regions, the central region having one or more first pressure openings formed through an exterior surface of the central region, the one or more first pressure openings configurable to provide an outward positive pressure, wherein the proximal region, central region, and distal region are collectively formed in such a way as to have a common central axis; and
an expandable member secured to the main assembly body, the expandable member being a rectangular sheet having a first end, second end opposite to the first end, third end, fourth end opposite to the third end, an inward facing interior surface, and an outward facing exterior surface facing opposite to the inward facing interior surface, wherein:
the inward facing interior surface of the expandable member faces the exterior surface of the central region of the main assembly body in such a way that the one or more first pressure openings face the inward facing interior surface of the expandable member;
the first end of the expandable member is secured to the proximal region of the main assembly body; and
the second end of the expandable member is secured to the distal region of the main assembly body;
wherein, when the one or more first pressure openings provide an outward positive pressure, the expandable member between the first and second ends of the expandable member is configured to expand outwardly away from the central region of the main assembly body.

30. The endoscopic system of claim 29, wherein the third and fourth ends of the expandable member are secured to one another.

31. The endoscopic system of claim 29, wherein one or more of the following apply:
the third end of the expandable member is secured to the central region of the main assembly body; and/or
the fourth end of the expandable member is secured to the central region of the main assembly body; and/or
the proximal region of the main assembly body includes one or more second pressure openings, the one or more second pressure openings configurable to provide an inward negative pressure.
